(19) United States
(12) Patent Application Publication
(10) Pub. No.: US 2023/0104050 A1
(43) Pub. Date: Apr. 6, 2023

(54) INDUCING PRODUCTION OF ANTI-OLIGOMANNOSE ANTIBODIES

(71) Applicant: SRI International, Menlo Park, CA (US)

(72) Inventors: Denong Wang, Palo Alto, CA (US); Lisa Wu, San Francisco, CA (US)

(73) Assignee: SRI International, Menlo Park, CA (US)

(21) Appl. No.: 17/907,313

(22) PCT Filed: Feb. 12, 2021
(86) PCT No.: PCT/US2021/017821

§ 371 Date: Sep. 26, 2022

Related U.S. Application Data
(60) Provisional application No. 63/009,045, filed on Apr. 13, 2020.

(51) Int. Cl. A61K 47/56; A61K 9/00; C07K 16/44; G01N 33/68

(52) U.S. Cl. CPC A61K 47/56; A61K 9/0019; C07K 16/44; G01N 33/6854; A61K 2039/58

(57) ABSTRACT

Example methods comprise administering an immunogenic vaccine composition to a subject, the immunogenic vaccine composition comprising a glycoconjugate. The method can further comprise, in response to the administration of the immunogenic vaccine composition, inducing production of anti-oligomannose antibodies in the subject and thereby eliciting an immune response to a viral pathogen in the subject.

### GOVERNMENT RIGHTS

This invention was made with government support under contract numbers R21AI124068-01A1 and R56AI118464-01A1 awarded by the National Institute of Allergy and Infectious Diseases of the National Institutes of Health and award number W81XWH-18-1-0604, awarded by the U.S. Army Medical Research Acquisition Activity. The government has certain rights in the invention.

### BACKGROUND

Viral diseases are infections caused by viral pathogens, also called viruses, a type of microorganism. There are many different types of viral pathogens that can cause a variety of different diseases. Viral diseases are contagious and can spread from one subject to another when the viral pathogen enters the body of the subject. If the immune system of the subject cannot fight off the virus, the virus can multiply and spread to other cells.

### SUMMARY

The present invention is directed to overcoming the above-mentioned challenges and others related to viral pathogens.

Various embodiments of the present disclosure are directed to a method comprising administering an immunogenic vaccine composition to a subject, the immunogenic vaccine composition comprising a glycoconjugate. The method further comprises, in response to the immunogenic vaccine composition, inducing production of anti-oligomannose antibodies in the subject and thereby eliciting an immune response to a viral pathogen in the subject. The viral pathogen can express and surface-expose oligomannoses that are associated with the glycoconjugate.

In some embodiments, the method includes triggering splenic B-cell responses and co-activating C-type lectin dendritic cell-specific intercellular adhesion molecule-3-grabbing non-integrin (DC-SIGN)-mediated dendritic cell responses in response to the immunogenic vaccine composition, thereby inducing the production of the anti-oligomannose antibodies and eliciting the immune response.

In some embodiments, the anti-oligomannose antibodies include broadly neutralizing antibodies (bnAbs) that recognize surface-exposed oligomannoses expressed by the viral pathogen.

In some embodiments, administering the immunogenic vaccine composition includes injecting a soluble form of the immunogenic vaccine composition to the patient (e.g., a soluble glycoconjugate).

In some embodiments, administering the immunogenic vaccine composition includes administering a dosage range of the immunogenic vaccine composition to a subject (e.g., between 0.2 milligrams(mg)/kilogram(kg) to 0.3 mg/kg, or between 0.1 to 100.0 micrograms (µg) of the immunogenic vaccine composition).

In some embodiments, the method can further include administering an additional immunogenic vaccine composition to the subject, wherein the additional immunogenic vaccine composition comprises one of the glycoconjugate of the immunogenic vaccine composition and a different glycoconjugate from the glycoconjugate of the immunogenic vaccine composition. For example, each of the immunogenic vaccine composition and the additional immunogenic vaccine composition can include high mannose compositions. In some embodiments, the additional immunogenic vaccine composition can trigger splenic B-cell responses and co-activate C-type lectin DC-SIGN-mediated dendritic cell responses, and the immunogenic vaccine composition can further trigger splenic B-cell responses (and optionally boost co-activating C-type lectin DC-SIGN-mediated dendritic cell responses) and in response induce production of anti-oligomannose antibodies.

In some embodiments, administering the additional immunogenic vaccine composition to the subject includes administering a dosage range of between 0.2 mg/kg to 0.3 mg/kg or between 0.1 to 100.0 µg of the additional immunogenic vaccine composition to the subject, where the additional dose can either be the same or different dosage as the first dosage of the immunogenic vaccine composition administered.

In some embodiments, the glycoconjugate includes terminal glyco-epitopes recognized by the anti-oligomannose antibodies. In some embodiments, the glycoconjugate includes internal chain or side-face glyco-epitopes recognized by the anti-oligomannose antibodies.

In some embodiments, the method includes eliciting the immune response in the subject against the viral pathogen, the viral pathogen including at least one of Middle East respiratory syndrome coronavirus (MERS-CoV), severe acute respiratory syndrome (SARS)-CoV, SARS-CoV-2, Zika virus (ZIKV), Dengue virus (DENV), West Nile virus (WNV), human cytomegalovirus (HCMV), and Human immunodeficiency virus (HIV-1).

In some embodiments, the glycoconjugate includes a carrier protein linked to oligomannose chains having a plurality of glyco-epitopes recognized by the anti-oligomannose antibodies.

In some embodiments, the method further includes identifying oligomannoses for the glycoconjugate by screening the viral pathogen or a neutralizing agent that reacts with the viral pathogen against an array of a plurality of different oligomannoses.

In some embodiments, the method further includes, after eliciting the immune response, producing hybridomas using antibody producing cells obtained from the subject, screening the hybridomas for the anti-oligomannose antibodies using an array of a plurality of different oligomannoses, and generating oligomannose-specific mAbs from at least one of the anti-oligomannose antibodies.

In some embodiments, eliciting the immune response includes broadly providing prevention from infection or an immune response to different viral pathogens by the production of the anti-oligomannose antibodies in the subject, wherein each of the different viral pathogens express and surface-expose oligomannoses.

Various embodiments are directed to a method comprising administering a first immunogenic vaccine composition to a subject and administering a second immunogenic vaccine composition to the subject, the first and second immunogenic vaccine compositions each comprising a glycoconjugate having glyco-epitopes. The method further includes, in response, inducing production of anti-oligomannose antibodies, such as bnAbs that recognize the glyco-epitopes and thereby eliciting an immune response to a viral pathogen that expresses surface-exposed oligomannoses associated with the glyco-epitopes in the subject.

In some embodiments, administering the first and second immunogenic vaccine compositions includes providing a first intravenous injection to the subject that includes a soluble form of the first immunogenic vaccine composition, and in response, resulting in at least one of triggering splenic B-cell responses and co-activating the C-type lectin DC-SIGN-mediated dendritic cell responses. Administering the first and second immunogenic vaccine compositions can further include providing a second intravenous injection to the subject that includes a soluble form of the second immunogenic vaccine composition, and in response, boosting splenic B-cell responses and C-type DC-SIGN-mediated dendritic cell responses, thereby inducing the production of the anti-oligomannose bnAbs or other anti-oligomannose antibodies and eliciting the immune response. The immune response may be long-lasting.

In some embodiments, the first immunogenic vaccine composition and the second immunogenic vaccine composition include the same glycoconjugate having the same terminal glyco-epitopes. In some embodiments, the first immunogenic vaccine composition and the second immunogenic vaccine composition include different glycoconjugates having different terminal glyco-epitopes from another.

In some embodiments, eliciting the immune response includes broadly providing preventative infection from or immune response to different viral pathogens by the production of the anti-oligomannose bnAbs or other anti-oligomannose antibodies in the subject, wherein each of the different viral pathogens express and surface-expose oligomannoses. In some embodiments, eliciting the immune response includes providing preventative immune response to SARS-CoV and/or SARS-CoV-2.

Some embodiments are directed to a method comprising producing hybridomas using antibody producing cells (e.g., B-cells) obtained from a subject treated with an immunogenic vaccine composition, the immunogenic vaccine composition comprising a glycoconjugate designed to induce production of anti-oligomannose antibodies that recognize surface-exposed oligomannoses. The method further includes screening the hybridomas for the anti-oligomannose antibodies using an array of a plurality of different oligomannoses, and generating oligomannose-specific mAbs from at least one of the anti-oligomannose antibodies.

In some embodiments, generating the oligomannose-specific mAbs includes generating mAbs specific to one or more mannosyl moieties containing Man1, Man2, Man3, Man4, Man5, Man6, Man7, Man8, or Man9.

A number of embodiments are directed to an immunogenic vaccine composition for inducing production of anti-oligomannose antibodies in the subject and thereby eliciting an immune response to a viral pathogen in the subject, the immunogenic vaccine composition comprising a soluble glycoconjugate. In some embodiments, the anti-oligomannose antibodies recognize surface-exposed oligomannoses.

In some embodiments, the immunogenic vaccine composition triggers splenic B-cell responses and co-activates C-type lectin DC-SIGN-mediated dendritic cell responses, thereby inducing the production of the anti-oligomannose antibodies and eliciting the immune response.

Some embodiments are directed to an immunogenic vaccine composition. The immunogenic vaccine composition may comprise a carrier protein and a plurality of a first oligomannose chains linked to the carrier protein. The plurality of first oligomannose chains may include a plurality of glyco-epitopes recognized by anti-oligomannose antibodies. The carrier protein and plurality of first oligomannose chains may form a glycoconjugate. In some embodiments, each of the plurality of first oligomannose chains include one or more mannosyl moieties containing Man1, Man2, Man3, Man4, Man5, Man6, Man7, Man8, and/or Man9. In some embodiments, each of the plurality of first oligomannose chains include one or more mannosyl moieties of Man5. In some embodiments, each of the plurality of first oligomannose chains include one or more mannosyl moieties of Man9. Each of the plurality of first oligomannose chains may include a GlcNac₂ core structure and mannose monomers. The GlcNac₂ core structure may be linked to an amino acid (e.g., Asn) that is crosslinked to surface components (e.g., lysines) of the carrier protein. For example, the carrier protein may be selected from bacteriophage Qbeta (Qβ) and keyhole limpet hemocyanin (KLH), and each of the first oligomannose chains may include an Asn-(GlcNac)₂ linked high mannose glycan, such as one or more mannosyl moieties containing Man1, Man2, Man3, Man4, Man5, Man6, Man7, Man8, and/or Man9.

Some embodiments are directed to an immunogenic vaccine composition that comprises a carrier protein, a plurality of a first oligomannose chains linked to the carrier protein, and a plurality of second oligomannose chains linked to the carrier protein. The plurality of first oligomannose chains and the plurality of second oligomannose chains may include a plurality of glyco-epitopes recognized by anti-oligomannose antibodies. The carrier protein, the plurality of first oligomannose chains, the plurality of second oligomannose chains may form a glycoconjugate. In some embodiments, the plurality of first oligomannose chains and the plurality of second oligomannose chains may respectively include different terminal mannosyl moieties. For example, each of the plurality of first oligomannose chains may include one or more mannosyl moieties containing Man1, Man2, Man3, Man4, Man5, Man6, Man7, Man8, and/or Man9. And, each of the plurality of second oligomannose chains may include one or more mannosyl moieties containing Man1, Man2, Man3, Man4, Man5, Man6, Man7, Man8, and/or Man9, and that are different from the mannaosyl moieties of the first oligomannose chains. In some embodiments, each of the plurality of first oligomannose chains include one or more mannosyl moieties of Man5. In some embodiments, each of the plurality of second oligomannose chains include one or more mannosyl moieties of Man9. Each of the plurality of first and second oligomannose chains may include a GlcNac₂ core structure and mannose monomers. The GlcNac₂ core structure may be linked to an amino acid that is crosslinked to surface components of the carrier protein. For example, the carrier protein may be selected from bacteriophage Qβ and KLH, and each of the first and the second oligomannose chains may include an Asn-(GlcNac)₂ linked high mannose glycan, such as one or more mannosyl moieties containing Man1, Man2, Man3, Man4, Man5, Man6, Man7, Man8, and/or Man9.

### BRIEF DESCRIPTION OF THE DRAWINGS

Various example embodiments can be more completely understood in consideration of the following detailed description in connection with the accompanying drawings, in which:

FIG. 1 illustrates an example method of inducing production of anti-oligomannose antibodies, in accordance with the present disclosure.

FIG. 2 illustrates an example of inducing production of anti-oligomannose antibodies and eliciting an immune response, in accordance with the present disclosure.

FIG. 3 illustrates an example method of inducing production of anti-oligomannose antibodies, in accordance with the present disclosure.

FIG. 4 illustrates an example method of generating oligomannose-specific monoclonal antibodies (mAbs), in accordance with the present disclosure.

FIG. 5 illustrates an example array of oligomannoses, in accordance with present disclosure.

FIGS. 6A-6B illustrate a schematic of highly conserved cellular N-glycosylation pathway catalyzed by a series of glyco-gene products, in accordance with the present disclosure.

FIGS. 7A-7D illustrate example schematics of a synthetic glycoconjugate approach for distinct models of virus-neutralization, in accordance with the present disclosure.

FIG. 8 illustrates example induction of active IgG responses to oligomannose-based cryptic glyco-epitopes by an immunization strategy, in accordance with the present disclosure.

FIGS. 9A-9D illustrate example schematics of oligomannose-series of vaccine conjugates and a bacteriophage Qβ-control vector, in accordance with the present disclosure.

FIGS. 10A-10C illustrate example binding profiles for mannose-reactive proteins 2G12, Galanthus nivalis agglutinin (GNA), and Narcissus pseudonarcissus lectin (NPA) to oligomannose-Bovine serum albumin (BSA) conjugates, in accordance with the present disclosure.

FIGS. 11A-11D illustrate example schematics of oligomannose-series of vaccine conjugates and a Qβ-control vector, in accordance with the present disclosure.

FIGS. 12A-12B illustrate examples induction of active IgG responses to oligomannose-based virus-neutralizing epitopes by an immunization strategy, in accordance with the present disclosure.

FIGS. 13A-13B illustrate example mAbs that may recognize distinct epitopes of high-mannose antigens, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

Viral diseases can lead to serious complications, including hospitalization and death. Strategic global or regional healthcare responses to viral pathogens, such as novel or emerging viruses, can be used for protecting humans or other mammalian populations. However, different viral pathogens have different molecular targets or epitopes for neutralization. Developing vaccines against emerging viral pathogens requires versatile molecular targets for eliciting broadly neutralizing antibodies (bnAbs), and has been challenging due to the genetic diversity of infectious viruses. Embodiments in accordance with the present disclosure are directed to methods of administering immunogenic vaccines that can provide broad-spectrum protection against diverse groups of viral pathogens by targeting oligomannoses found on the surface of proteins of the viral pathogen. Such multi-purpose vaccines can be used against the emergence of novel, or re-emergence of unexpected, viral pathogens.

Viral pathogens can decorate their outer surfaces with oligomannoses, a type of glycan. Many viral pathogens do not have mechanisms for glycosylation and depend on host cells of the subject for glycosylation. For example, protein surfaces of the viral pathogen can be covered in the oligomannoses produced by the host cell, which can limit antibody access to the protein neutralizing epitopes of the viral pathogen. The oligomannoses belong to a class of N-glycan cryptic autoantigens with unique immunological properties. The oligomannoses are generally present intracellularly as glycosylation intermediates, and can become overexpressed and/or surface-exposed by some viral pathogens, as well as tumor cells. As the oligomannoses are generally present intracellularly as glycosylation intermediates, induction of immune responses to oligomannose targets is unlikely to be harmful to normal cells. For example, oligomannose targets in accordance with the present disclosure, such as oligomannosly glycans, can be used to target viral pathogens without, or with minimizing, impact to normal cells which present the glycosylation intermediates intracellularly. As an example, antibodies or lectins targeting the cryptic intracellular antigens (e.g., the oligomannoses) can be used for clearance of autoantigens released from the aged or apoptotic cells in vivo.

Example embodiments in accordance with the present disclosure are directed to methods of eliciting an immune response in a subject to a viral pathogen. Example methods include administering an immunogenic vaccine composition to a subject. The immunogenic vaccine composition can include a glycoconjugate having glyco-epitopes. The immunogenic vaccine composition can trigger splenic B-cell responses and co-activate C-type lectin DC-SIGN-mediated dendritic cell responses, thereby inducing the production of the anti-oligomannose antibodies and eliciting the immune response to the viral pathogen that expresses and/or surface-exposes the glyco-epitopes. In some embodiments, the method can include administering a first immunogenic vaccine composition to a subject and administering a second immunogenic vaccine composition to the subject, where the first and second immunogenic vaccine compositions each comprising a glycoconjugate having glyco-epitopes.

Some embodiments are directed to a method that includes producing hybridomas using antibody producing cells obtained from a subject treated with an immunogenic vaccine composition, the immunogenic vaccine composition comprising a glycoconjugate designed to induce production of anti-oligomannose antibodies that recognize surface-exposed oligomannoses. The method further includes screening the hybridomas for the anti-oligomannose antibodies using an array of a plurality of different oligomannoses, and generating oligomannose-specific mAbs from at least one of the anti-oligomannose antibodies.

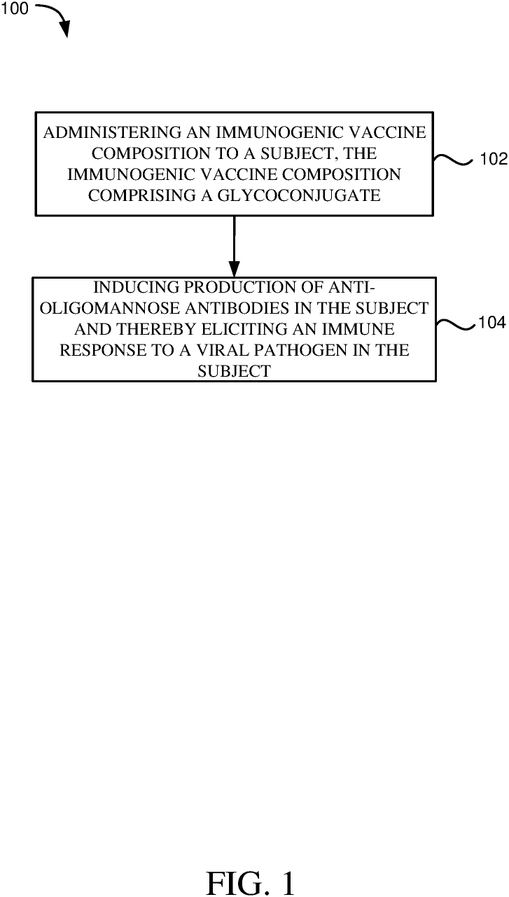

Turning now to the figures, FIG. 1 illustrates an example method of inducing production of anti-oligomannose antibodies, in accordance with the present disclosure.

At 102, the method 100 includes administering an immunogenic vaccine composition to a subject. The subject can include a mammal, such as a human, a monkey, a mouse, a pig, a cow, a ferret, a hamster, a dog, a cat, among other types of mammals. In some embodiments, administering the immunogenic vaccine composition includes injecting a soluble form of the immunogenic vaccine composition.

In some embodiments, administering the immunogenic vaccine composition to the subject includes administering a dosage range of the immunogenic vaccine composition to the subject, e.g., such as a dosage range between 0.2 mg/kg to 0.3 mg/kg, wherein 0.2 mg/kg refers to 0.2 mg of the immunogenic vaccine composition per kg of the subject. In some embodiments, the dosage includes 0.25 mg/kg. In some embodiments, the dosage range may be between 0.1 to 100.0 µg of the immunogenic vaccine composition and/or the glycoconjugate. In some embodiments, different types of mammals may be provided a dosage range of between 0.2 mg/kg to 0.3 mg/kg from mammals provided a dosage range of between 0.1 to 100.0 µg of the immunogenic vaccine composition. In some embodiments, a dosage range of between 0.2 mg/kg to 0.3 mg/kg may be used for smaller mammals, such as mice. In some embodiments, a dosage range of between 0.1 to 100.0 µg may be used for larger mammals, such as humans. However embodiments are not so limited, and the above provided range is provided as a non-limiting example of experimental vaccine doses, which may be for mammals.

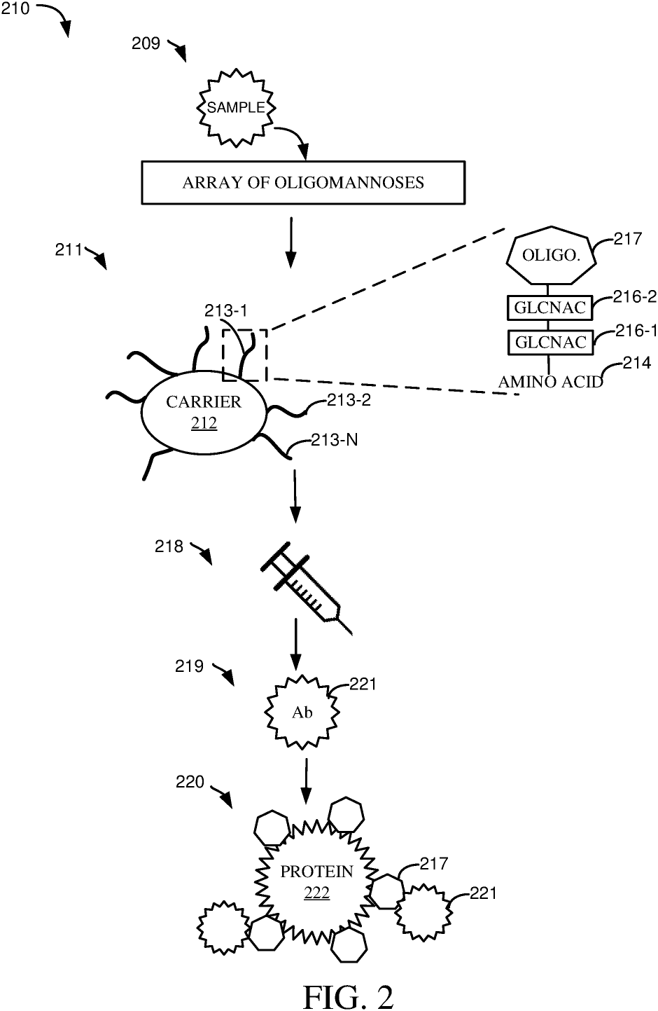

The immunogenic vaccine composition can comprise a glycoconjugate. For example, the immunogenic vaccine composition can include a soluble glycoconjugate that is injectable. In some embodiments, the glycoconjugate can include glyco-epitopes (e.g., sugar moieties) recognized by the anti-oligomannose antibodies. As further illustrated and described herein at least in association with FIG. 2, the glycoconjugate can include a carrier protein linked to oligomannose chains having a plurality of glyco-epitopes (e.g., terminal and, in some instances, internal or side-facing) recognized by the anti-oligomannose antibodies. Example oligomannose chains can include mannosyl moieties containing one or more of Man1, Man2, Man3, Man4, Man5, Man6, Man7, Man8, and Man9. In various embodiments, oligomannose chains in the glycoconjugate are not limited to pure oligomannoses, and may include hybrid and/or complex forms of oligomannoses. The anti-oligomannose antibodies may not, or may have minimal, impact on normal cells as the anti-oligomannose antibodies are specific to oligomannoses which are generally present intracellularly as glycosylation intermediates by the normal cells, as discussed above.

In some embodiments, the method includes triggering splenic B-cell responses and co-activating C-type lectin DC-SIGN-mediated dendritic cell responses in response to the immunogenic vaccine composition, thereby inducing the production of the anti-oligomannose antibodies and eliciting the immune response. For example, Man9 is a ligand for DC-SIGN and can be used in the glycoconjugate of the immunogenic vaccine composition to both trigger B-cell responses and co-activate C-type lectin DC-SIGN-mediated dendritic cell responses, and thereby induce the production of the anti-Man9 antibodies in the subject. In some embodiments, the glycoconjugate of the immunogenic vaccine composition includes a combination of Man9 and another oligomannose, such as Man5 which triggers B-cell responses and thereby induces the production of the anti-Man5 antibodies in the subject.

In some embodiments, the method 100 can include identifying oligomannoses for the glycoconjugate by screening the viral pathogen or a neutralizing agent that reacts with the viral pathogen against an array of a plurality of different oligomannoses. The array of the plurality of different oligomannoses can include an array of different oligomannoses that include moieties containing Man1, Man2, Man3, Man4, Man5, Man6, Man7, Man8, and/or Man9, sometimes herein generally referred to as a “carbohydrate panel”. The neutralizing agent can include antibodies or lectins of known oligomannose-binding specificities or anti-serum obtained from a viral infected or vaccinated subjects.

In some embodiments, the method 100 can further include administering an additional immunogenic vaccine composition to the subject. The additional immunogenic vaccine composition may be administered at a different time from the immunogenic vaccine composition, such as prior to administering the immunogenic vaccine composition. In some such embodiments, the additional immunogenic vaccine composition can include the same glycoconjugate or a different glycoconjugate from the glycoconjugate of the immunogenic vaccine composition. For example, each of the immunogenic vaccine composition and the additional immunogenic vaccine composition can include a high mannose composition that is administered to the subject to trigger B-cell responses and induce production of antibodies specific to one or more oligomannoses. In some embodiments, the additional immunogenic vaccine composition is a DC-SIGN-reactive oligomannose (such as containing Man9 or other DC-SIGN ligands) that triggers B-cell responses and co-activation of C-type lectin DC-SIGN-mediated dendritic cell responses. In some embodiments, the additional immunogenic vaccine composition is a high mannose composition that is administered to the subject to trigger B-cell responses and induce production of antibodies specific to Man5 and/or specific to Man9. In some embodiments, the additional immunogenic vaccine composition includes multiple mannosyl moieties, such as Man5 and Man9 or other combinations. In some embodiments, administering the additional immunogenic vaccine composition to the subject includes administering a dosage range of between 0.2 mg/kg to 0.3 mg/kg of the additional immunogenic vaccine composition to the subject, where the dose of the additional immunogenic vaccine composition can either be the same or different dosage as the dose of the immunogenic vaccine composition.

At 104, in response to the immunogenic vaccine composition, the method 100 includes inducing production of anti-oligomannose antibodies in the subject and thereby eliciting an immune response to a viral pathogen in the subject. Anti-oligomannose antibodies, as used herein, include and/or refer to antibodies that recognize oligomannoses, such as by binding to terminal glyco-epitopes and/or internal chains or side face glyco-epitopes of the oligomannoses. In some embodiments, the anti-oligomannose antibodies recognize the native forms of oligomannoses, such as oligomannosyls in a native glycoprotein, glycolipid, or other forms of carbohydrate-containing macromolecules. The particular oligomannoses can be associated with one or more viral pathogens. For example, the particular oligomannoses can be expressed and surface-exposed by the viral pathogen. In some embodiments, different viral pathogens can use the same one or more oligomannoses as a surface shield, thereby exhibiting the one or more oligomannoses on the surface of the viral protein. In some embodiments, the anti-oligomannose antibodies include bnAbs. The bnAbs can recognize the surface-exposed oligomannoses expressed by the viral pathogen or pathogens.

As used herein, bnAbs include and/or refer to antibodies which can neutralize (e.g., kill) multiple viral pathogens or multiple viral pathogen strains. In various embodiments, the bnAbs target conserved epitopes of surface-exposed oligomannoses associated with the virus, which may not change between different mutations of a virus and/or are used by different viral pathogens. Eliciting the protective immune response can thereby include broadly providing prevention from infection or an immune response to different viral pathogens by the production of the anti-oligomannose antibodies in the subject. For example, the bnAbs can be used to elicit an immune response to variety of different viral pathogens. Each of the different viral pathogens can express and surface-expose oligomannoses which exhibit the glyco-epitopes. In some embodiments, the immune response is elicited in the subject against the viral pathogen selected from MERS-CoV, SARS-CoV, SARS-CoV-2, HCMV, ZIKV, DENV, WNV, and HIV-1, and combinations thereof.

In some embodiments, the method 100 can further include generating oligomannose-specific mAbs. For example, after eliciting the immune response, the method 100 can further include producing hybridomas or immobilized (and live) antibody producing cells using antiserum obtained from the subject, and screening the hybridomas or the antibody producing cells for the anti-oligomannose antibodies using an array of a plurality of different oligomannoses. The method can further include generating oligomannose-specific mAbs from at least one of the anti-oligomannose antibodies, as further described herein.

FIG. 2 illustrates an example method of inducing production of anti-oligomannose antibodies and eliciting an immune response to a viral pathogen, in accordance with the present disclosure. The method 210 can include an example implementation of the method 100 as illustrated by FIG. 1.

As shown at 209, an immunogenic vaccine composition can be selected that comprises a glycoconjugate. The immunogenic vaccine composition can be selected by screening the viral pathogen and/or a neutralizing agent from a subject infected with the viral pathogen. For example, a neutralizing agent can be screened against an array of a plurality of different oligomannoses, and used to identify which of the different oligomannoses binds to the neutralizing agent. The identified one or more oligomannoses can be used to generate the immunogenic vaccine composition.

As shown at 211, the glycoconjugate can include a carrier protein 212 linked to a plurality of oligomannose chains 213-1, 213-2, 213-N having a plurality of glyco-epitopes recognized by anti-oligomannose antibodies. The plurality of plurality of oligomannose chains 213-1, 213-2, 213-N can form one or more oligomannose clusters. As shown by the particular oligomannose chain 213-1, in some embodiments, the oligomannose chains can each include an N-linked oligosaccharide having an N-Acetylglucosamine (GlcNAc)₂-linked core structure. For example, the GlcNAc₂-linked core structure can include an amino acid 214 -GlcNAc₂ 216-1, 216-2, such as asparagine (Asn)-(GlcNAc)₂. The amino acid 214 -(GlcNAc)₂ 216-1, 216-2 can be linked to a high mannose glycan, e.g., the particular oligomannose 217, such as an oligomannose having a mannosyl moiety selected from Man1, Man2, Man3, Man4, Man5, Man6, Man7, Man8, Man9.

In some embodiments, the plurality of oligomannose chains 213-1, 213-2, 213-N can be linked to surface components of the carrier protein 212. For example, the GlcNac₂ core structures can be linked to an amino acid (e.g., Asn) and the amino acid can be crosslinked to surface components of the carrier protein 212, such as lysine residues of the carrier protein 212. Non-limiting examples of carrier proteins include bacteriophage Qbeta (Qβ) and keyhole limpet hemocyanin (KLH).

In some embodiments, the oligomannose and/or a plurality of different oligomannoses can be used in the immunogenic vaccine composition. In some embodiments, the carrier protein 212 can have a plurality of each of two or more different oligomannose chains.

Each of the different oligomannose chains can have different glyco-epitopes which are recognized by the same or different anti-oligomannose antibodies. For example, a first oligomannose chain can be linked to the carrier protein 212 and a second oligomannose chain can be linked to the carrier protein 212, where the first oligomannose chain and the second oligomannose chain include a plurality of glyco-epitopes recognized by anti-oligomannose antibodies. In some embodiments, the first oligomannose chain and the second oligomannose chain respectively include different terminal glyco-epitopes (e.g., terminal sugar moieties). In some embodiments, each of the first and second (or more) oligomannose chains can include mannosyl moieties selected from Man1, Man2, Man3, Man4, Man5, Man6, Man7, Man8, and Man9, where the first and second oligomannose chains are different from one another. As an example, the first oligomannose chain can include Man5 and the second oligomannose chain can include Man9. Man9 can include a high affinity ligand for C-type lectin, such as DC-SIGN. Man5 displays a potent GNA-epitope for eliciting bnAbs responses.

In some embodiments, example methods include forming the immunogenic vaccine composition. For example, a method can include linking a plurality of a first oligomannose chain to the carrier protein 212, and linking a plurality of a second oligomannose chain to the carrier protein 212, thereby forming an immunogenic vaccine composition having a plurality of different glyco-epitopes recognized by anti-oligomannose antibodies. Similarly to that described above, such example methods can include, synthetically forming the plurality of first and second oligomannose chains using a GlcNac₂ core structure and mannose monomers. In some embodiments, the first oligomannose chains and the second oligomannose chains include different terminal glyco-epitopes (e.g., sugar moieties).

At 218, the method 210 includes administering the immunogenic vaccine composition to a subject by providing an intravenous (IV) injection. As previously described, the immunogenic vaccine composition can include a soluble glycoconjugate. In some embodiments, a single IV injection is provided. In some embodiments, two IV injections are provided at different times, and which can include the same or different immunogenic vaccine compositions.

At 219, in response, the method includes inducing production of anti-oligomannose antibodies, such as the particular antibody 221, in the subject. And, at 220, the method 210 includes eliciting an immune response to a viral pathogen in the subject, such as to a protein 222 of the viral pathogen. For example, the viral protein 222 can express and expose oligomannoses on the surface of the protein 222, such as illustrated by the particular oligomannose 217. The particular oligomannose 217 can include terminal and/or internal glyco-epitopes (e.g., sugar moieties) recognized by the anti-oligomannose antibodies, such as the particular antibody 221. In some embodiments, the anti-oligomannose antibodies can bind to the particular oligomannose 217 and neutralize the viral pathogen.

In accordance with various embodiments, the anti-oligomannose antibodies can be specific to a particular viral pathogen. In some embodiments, the anti-oligomannose antibodies can be broadly neutralizing in that the anti-oligomannose antibodies can provide an immune response to a variety of different viral pathogens.

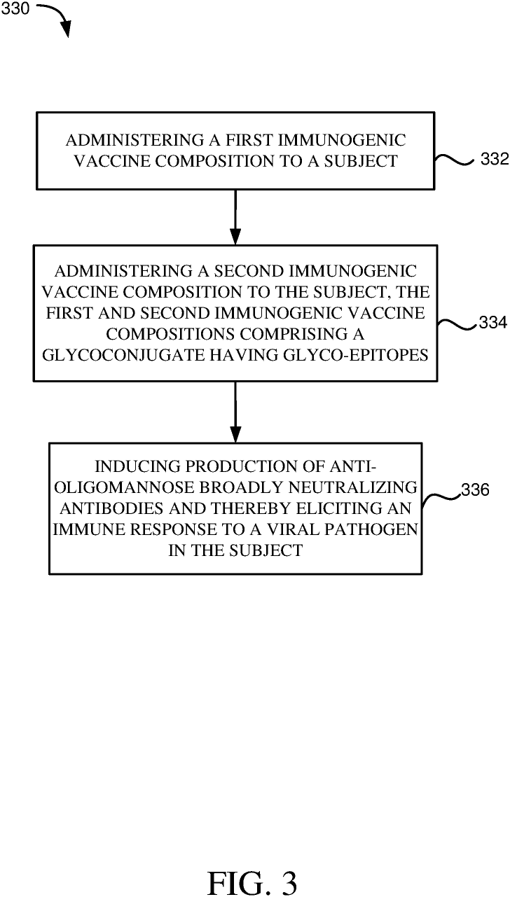

FIG. 3 illustrates an example method of inducing production of anti-oligomannose antibodies, in accordance with the present disclosure. In some examples, the method 330 can include an implementation of the method 100 and/or method 210 as illustrated by FIG. 1 and FIG. 2, respectively.

At 332, the method 330 includes administering a first immunogenic vaccine composition to a subject. At 334, the method includes administering a second immunogenic vaccine composition to the subject. The first and second immunogenic vaccine compositions can each comprise a glycoconjugate having glyco-epitopes. In some embodiments, administering the first and second immunogenic vaccine compositions can include providing a first IV injection to the subject that includes a soluble form of the first immunogenic vaccine composition, and providing a second IV injection to the subject that includes a soluble form of the second immunogenic vaccine composition. In some embodiments, the first IV injection can cause at least one of triggering splenic B-cell responses and co-activating the C-type lectin DC-SIGN-mediated dendritic cell responses, and the second IV inject can cause boosting of the splenic B-cell responses and C-type DC-SIGN-mediated dendritic cell responses, and thereby inducing the production of the anti-oligomannose bnAbs and eliciting the immune response, as further described below.

In some embodiments, the first and second immunogenic vaccine compositions can be administered to the subject at different times. For example, the second immunogenic vaccine composition can be administered a threshold amount of time after the first immunogenic vaccine composition is administered. In some embodiments, the threshold amount of time can include one week to six weeks. In some embodiments, the threshold amount of time can include two weeks to four weeks. In some embodiments, a dosage range of between 0.2 mg/kg to 0.3 mg/kg of the first immunogenic vaccine composition is administered to the subject, and a dosage range of between 0.2 mg/kg to 0.3 mg/kg of the second immunogenic vaccine composition is administered to the subject. In some embodiments, the dose of the first immunogenic vaccine composition can either be the same or different dosage as the dose of the second immunogenic vaccine composition. However, embodiments are not so limited and the range is provided as a non-limiting example. For example, in some embodiments, the dosage range may be between 0.1 to 100.0 µg of the first and/or second immunogenic vaccine compositions and/or the respective glycoconjugates, where the dose of the first immunogenic vaccine composition can either be the same or different dosage as the dose of the second immunogenic vaccine composition.

In some embodiments, the first immunogenic vaccine composition and the second immunogenic vaccine composition include the same glycoconjugate having the same terminal glyco-epitopes. In some embodiments, where the first immunogenic vaccine composition and the second immunogenic vaccine composition are the same, the concentration of glycoconjugate in the first immunogenic vaccine composition may be the same or different as compared to the concentration glycoconjugate in the second immunogenic vaccine composition. In some embodiments, first immunogenic vaccine composition and the second immunogenic vaccine composition include different glycoconjugates having different terminal glyco-epitopes from another. Similarly, the concentration of glycoconjugate in the first immunogenic composition may be the same or different as compared to the glycoconjugate concentration in the second immunogenic composition being administered.

At 336, the method 330 further includes, in response to the administrations of first and second immunogenic vaccine compositions, inducing production of anti-oligomannose bnAbs in the subject and thereby eliciting an immune response in the subject to a viral pathogen that express surface-exposed oligomannoses associated with the glyco-epitopes. The bnAbs can recognize the glyco-epitopes associated with the first and/or second immunogenic vaccine compositions. As described above, eliciting the immune response can include broadly providing preventative infection from or immune response to different viral pathogens by the production of the anti-oligomannose bnAbs in the subject, where each of the different viral pathogens express and surface-expose oligomannoses that include the glyco-epitopes.

In various embodiments, the immunogenic vaccine composition, such as the immunogenic vaccine composition described in association with FIG. 2 or the first and/or second immunogenic vaccine compositions described in association with FIG. 3 can include at least some of substantially the same features and/or attributes as described by Francesca Micoli, et al., “Protein Carriers for Glycoconjugate Vaccines: History, Selection Criteria, Characterization and Trends”, Molecules, 2018, Vol. 23, 1451; Rena D. Astronomo, “Defining Criteria for Oligomannose Immunogens for HIV using Icosahedral Virus Capsid Scaffolds”, Chemistry & Biology , 2010, Vol. 17, 357-350; Rena D. Astronomo, et al., “Carbohydrate Vaccines: Developing Sweet Solutions to Sticky Situations?”, Nature Reviews, Drug discovery, 2010, Vol. 9, 4, 308-24; and Sumati Bhatia, et al., “Multivalent Glycoconjugates as Vaccines and Potential Drug Candidates”, Med. Chem. Commun., 2015, Vol. 5, 862-878; each of which are fully incorporated herein by reference in their entirety for their teaching.

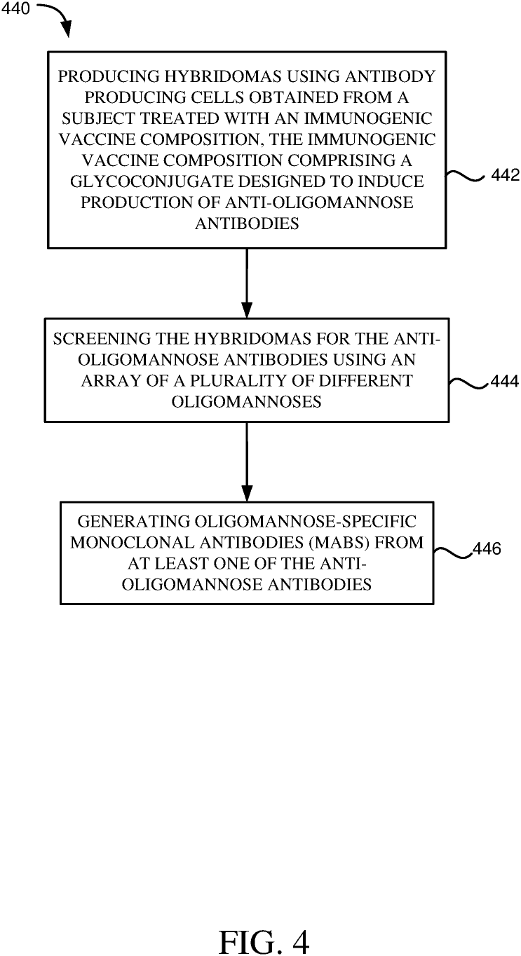

FIG. 4 illustrates an example method of generating oligomannose-specific mAbs, in accordance with the present disclosure. In some embodiments, the method 440 as illustrated by FIG. 4 can be used in combination with the methods 100, 210, 330 illustrated by FIGS. 1-3. However embodiments are not so limited.

At 442, the method 440 includes producing hybridomas using antibody producing cells obtained from a subject treated with an immunogenic vaccine composition, the immunogenic vaccine composition comprising a glycoconjugate designed to induce production of anti-oligomannose antibodies. The hybridomas can be produced by collecting antiserum from the subject and fusing an antibody-producing cell (e.g., a B-cell) with a myeloma cell to produce the hybridoma. As may be appreciated, hybridomas can be grown in culture, with each culture starting with a viable hybridoma cell, and producing cultures that include genetically identical hybridomas that produce one antibody per culture. In some examples, the hybridomas can be generated using at least some of substantially the same features and/or attributes as described in Chonghui Zhang C, “Hybridoma Technology for the Generation of Monoclonal Antibodies”, Antibody Methods and Protocols, Methods in Molecular Biology (Methods and Protocols), 2012, Vol. 901, Humana Press, which is fully incorporated herein by reference in its entirety for its teaching. However, embodiments are not so limited, and can include producing antibody-producing cells (e.g., a B-cell) used to generate anti-oligomannose antibodies with or without generating hybridomas.

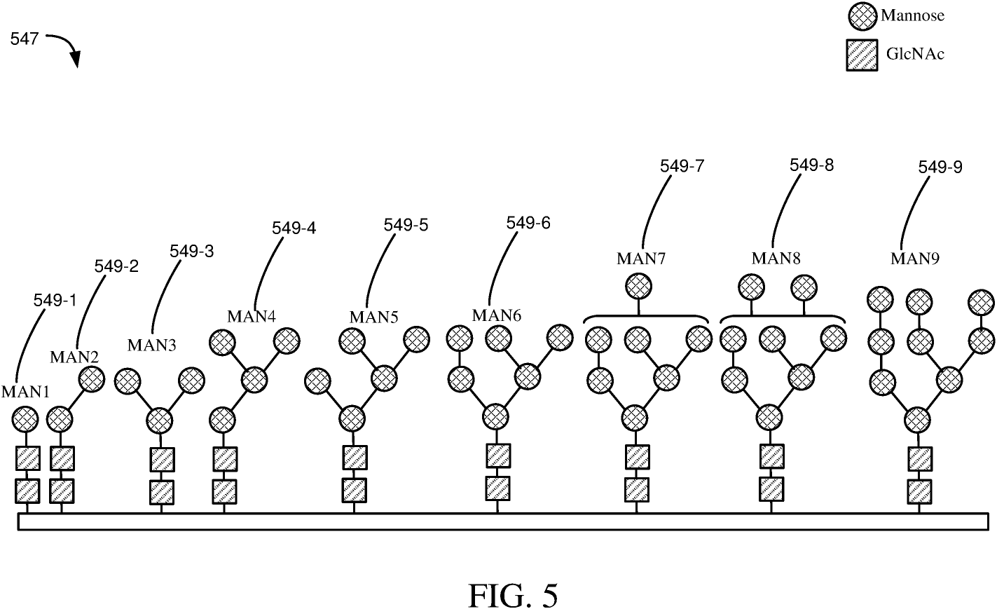

At 444, the method 440 includes screening the hybridomas for the anti-oligomannose antibodies using an array of a plurality of different oligomannoses (e.g., carbohydrate panel). An example array 547 is further illustrated by FIG. 5.

At 446, the method includes generating oligomannose-specific mAbs from at least one of the anti-oligomannose antibodies. In some embodiments, the oligomannose-specific mAbs can be specific to one or more of Man1, Man2, Man3, Man4, Man5, Man6, Man7, Man8, and Man9, such as unprocessed versions of oligomannoses. In some examples, the mAbs can be generated using at least some of substantially the same features and/or attributes as described in Irvin Y. Ho, et al., “Refined Protocol for Generating Monoclonal Antibodies from Single Human and Murine B Cells”, Journal of Immul. Methods, 2016, Vol. 438, 67-70, which is fully incorporated herein by reference in its entirety for its teaching.

FIG. 5 illustrates an example array of a plurality of different oligomannoses, in accordance with present disclosure. The array 547 can be used in the method 440 illustrated by FIG. 4 to identify anti-oligomannose antibodies and/or in the method 210 illustrated by FIG. 2 to identify oligomannoses specific to a particular viral pathogen. As shown, the array 547 includes a plurality of different oligomannoses 549-1, 549-2, 549-3, 549-4, 549-5, 549-6, 549-7, 549-8, 549-9. While the array 547 of FIG. 5 illustrates one of each of the different oligomannoses 549-1, 549-2, 549-3, 549-4, 549-5, 549-6, 549-7, 549-8, 549-9, the array may include a plurality of each of the different oligomannoses 549-1, 549-2, 549-3, 549-4, 549-5, 549-6, 549-7, 549-8, 549-9. In some examples, the different oligomannoses 549-1, 549-2, 549-3, 549-4, 549-5, 549-6, 549-7, 549-8, 549-9 include Man1, Man2, Man3, Man4, Man5, Man6, Man7, Man8, and Man9.

### EXPERIMENTAL EMBODIMENTS

A number of experimental embodiments were conducted to identify oligomannoses to use for a virus neutralizing or immunogenic vaccine composition, strategies for immunizing a subject using an immunogenic vaccine composition, as well as administering subjects with the composition and illustrating resulting anti-oligomannose antibodies. Some embodiments were directed to studying viral glycome and resulted in the recognition of a class of N-glycan cryptic glycan markers that are evolutionarily conserved among many mammalian viruses. A number of lectins that specifically bind to these sugar moieties, such as oligomannoses, are highly active in virus neutralization. A notable example is a plant-derived lectin, GNA, which cross-reacts with and effectively neutralizes viruses of distinct phylogenetic origins. However, lectins of non-human-origin are foreign antigens to humans and are not suitable for human therapeutic or prophylactic application in vivo.

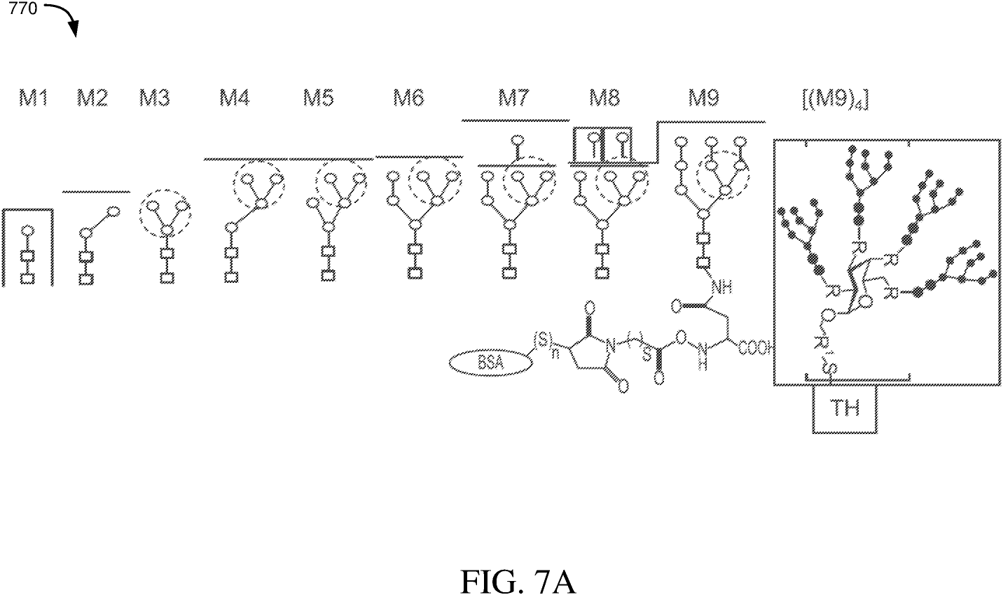

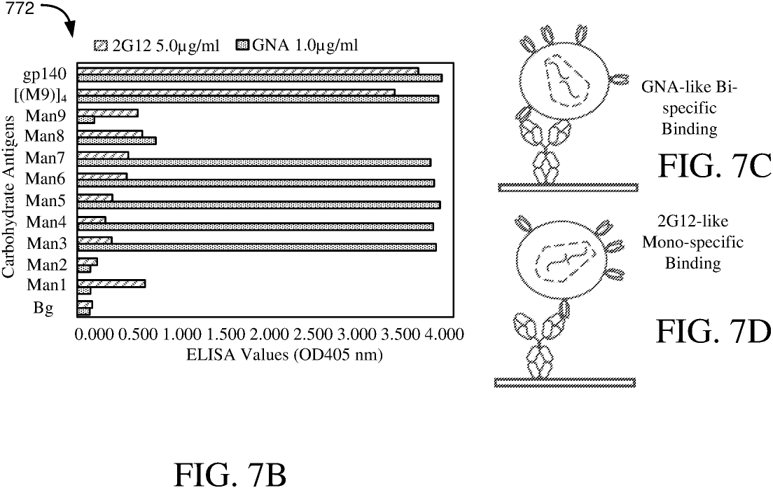

Various embodiments were directed to identification of the glyco-epitopes (e.g., sugar moieties) recognized by GNA and for use as versatile vaccine candidates to induce the protective immunity against emerging viral pathogens. The GNA-epitope-based vaccine strategy can elicit the GNA-like, anti-oligomannose antibodies, and these antibodies offer a GNA model of bi-specific cross-linking heteroligation of virions (e.g., see FIGS. 7C and 7D) and can function as bnAbs against emerging viral pathogens within mammals. As further shown, the glycoconjugate vaccine elicits the GNA-like antibodies in multiple strains of mice and the anti-sera bind to a broad-range of viruses. Furthermore, mAbs elicited by the vaccine can exhibit GNA-like glycan-binding characteristics and can effectively neutralize a range of emerging viral pathogens.

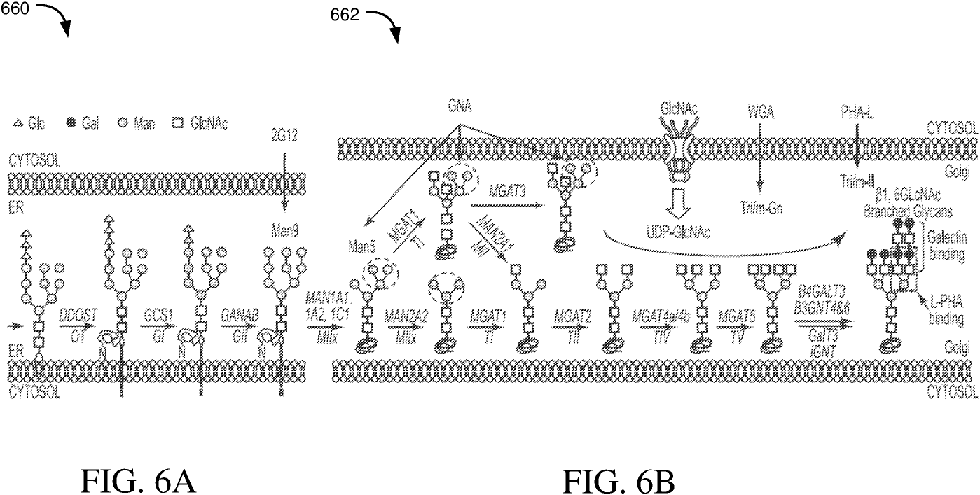

FIGS. 6A-6B illustrate a schematic of highly conserved cellular N-glycosylation pathway catalyzed by a series of glyco-gene products, in accordance with the present disclosure. As described above, virus particles are generally decorated with the host cell-derived oligomannoses. Mammalian viruses rely on host glycosylation machineries to synthesize glycans and are generally decorated with the host cell-derived oligomannoses.

More particularly, FIGS. 6A-6B illustrate an example of an N-glycosylation pathway in mammal cells which has the potential to generate numerous internal N-glycan chains, such as oligomannoses of varies of structural configurations, tri-antennary type II or multi-valent type II (Tri/m-II), and the agalactosyl derivative Tri/m-Gn (GlcNAc) antigens. These carbohydrates belong to a class of N-glycan cryptic autoantigens that are generally present intracellularly as glycosylation intermediates, but become overexpressed and/or surface-exposed by many viruses.

As shown by 660 and 662 in FIGS. 6A-6B, a number of N-glycan cryptic glycans produced by the host cell glycosylation pathway are found to be overexpressed and/or surface-exposed by viral pathogens. These include: a) oligomannoses recognized by virus-neutralizing agent 2G12, as shown by 660 in FIG. 6A, and GNA, as shown by the 662 in FIG. 6B, and b) agalacto moieties Tri/m-Gn recognized by Wheat Germ Agglutinin (WGA) and asialo-Tri/m-II epitopes by Phaseolus vulgaris-L lectin (PHA-L) and SARS-CoV neutralization antibodies, as shown by 662 in FIG. 6B.

Some embodiments were directed to constructing a large panel of glycoconjugates for identifying glyco-epitopes recognizes by GNA. GNA positive glycol-epitopes (e.g., sugar moieties) can be vaccine candidates to elicit bnAbs against emerging viral pathogens.

FIGS. 7A-7D illustrate a schematic of a synthetic glycoconjugate approach for distinct models of virus-neutralization, in accordance with the present disclosure.

FIG. 7A illustrates a panel of glycoconjugates 770 that was constructed to explore the potential glyco-epitopes recognized by GNA. Synthetic oligomannose clusters include Man1-Man9 are oligomannose-maleimide-BSA conjugates. [(Man9)4]-TH mimics the native HIV spike epitopes recognized by 2G12 and GNA. A dominant tri-mannose GNA epitope is marked by dashed circles. As used herein, Man is sometime interchangeably referred to as M, such as Man9 being referred to as M9.

FIG. 7B illustrates example graph 772 showing glycan-binding profiles in an enzyme-linked immunosorbent assay (ELISA) of the GNA and 2G12 to the panel of glycoconjugates 770 of FIG. 7A. To examine whether the synthetic carbohydrate antigens of the panel of glycoconjugates 770 reconstructed the native virus-neutralizing epitopes, the glycoconjugates were coated on ELISA micro-titer plates and then stained the plates with virus-neutralizing agent GNA (1.0 µg/mL) or 2G12 (5.0 µg/mL). A preparation of HIV Env protein was also applied to serve as a positive control, as shown by the ELISA values in the graph 772 of FIG. 7B. Oligomannose cluster preparations and HIV gp140 were coated on ELISA plates at 10 µg/mL and 2.0 µg/mL, respectively, and stained with 2G12 or GNA at specified concentrations. ELISA readouts of the two virus-neutralizing agents against a panel of 11 antigens are plotted in parallel to illustrate their glycan-binding profiles, respectively. The two virus-neutralizing agents were strongly reactive with the fully glycosylated HIV Env protein. However, they illustrate distinct patterns in reacting with the panel of oligomannose clusters. GNA is highly reactive with M3-M7 and [(Man9GlcNAc2Asn)₄]-T-helper peptide conjugate, which is labeled as (M9)₄-TH in FIGS. 7A-7B. By contrast, 2G12 is selectively reactive with [(M9)₄]-TH with minimal binding to other oligomannose antigens. The [(M9)₄]-TH conjugate was designed to mimic the high-density Man9 clusters of HIV envelope spikes, which are highly specific for HIV-bnAbs, such as 2G12 and some PGT-series of mAbs. The GNA epitopes are well-preserved by the tetra-Man9 conjugates.

FIGS. 7C and 7D illustrate two example models of virus-neutralization. FIG. 7C illustrates a GNA model of heteroligation and FIG. 7D illustrates a 2G12 model of homoligation binding of HIV virions. The two agents appear to interact with a virion in different ways, as shown by FIGS. 7C and 7D. Unlike 2G12, which is mono-specific for the tetra Man9 clusters, GNA recognizes a panel of oligomannose antigens with high affinity, as shown by FIG. 7D. As illustrated by FIG. 7C, the GNA-model of bi-specific binding involves use of one binding site for the epitope displayed by a virus surface spike and a second site on another viral epitope outside the spike. GNA can, thus, offer cross-linking “heteroligation” of viral surface oligomannose markers. By contrast, binding of the virion with 2G12-like mono-specific binding fails to cross-link HIV surface epitopes since 2G12 binds to the spike-epitope but not those outside the spike; the spikes are present at low density with only about 15 spikes per virion. Thus, the GNA model of glycan binding may offer a molecular mechanism to significantly increase the apparent affinity of virus binding and neutralization by the effective cross-linking of viral surface glyco-epitopes.

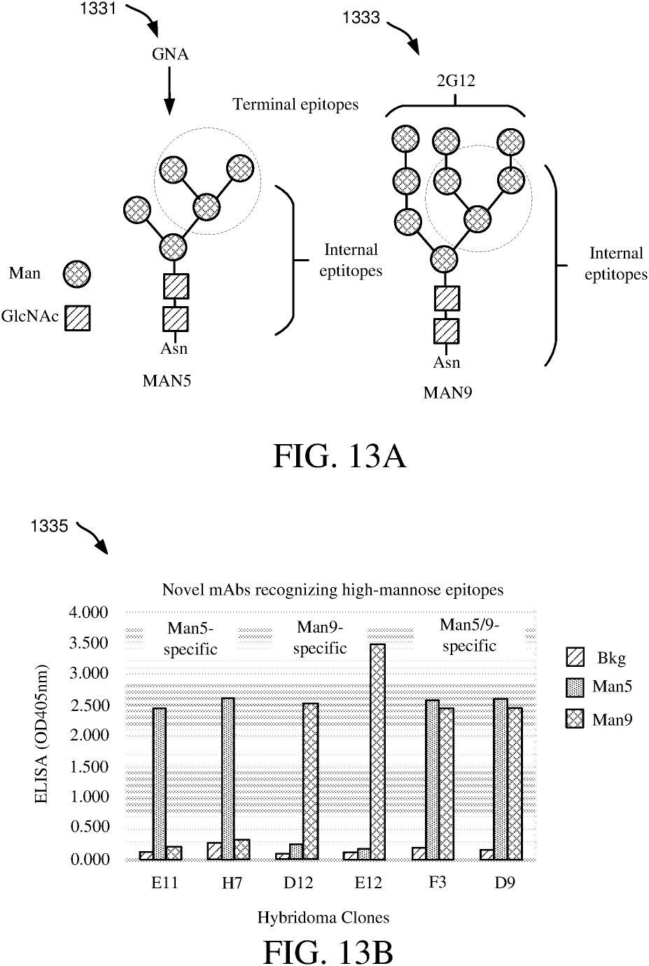

In various embodiments, a vaccine strategy was established to trigger active IgG responses to oligomannose antigens. Conceptually, the same oligosaccharide was displayed in different ways to direct the immune responses to the glyco-epitopes. In some embodiments, a representative GNA+ oligomannoside, Man5, was coupled to surface lysine residues on the icosahedral capsids of bacteriophage Qβ in different cluster configurations, e.g., Qβ-HIV gp120-V1V2 domain-AsnGlcNAc2Man5(Qβ-V1V2-M5), which was initially designed to present the HIV-specific PG9/16-epitopes, and Qβ-(AsnGlcNAc2Man9)/ AsnGlcNAc2Man5 (Qβ-M9/M5). Thus, the GNA+ M5-epitope was presented by both conjugates but in different structural configurations, see FIG. 13A for illustrations.

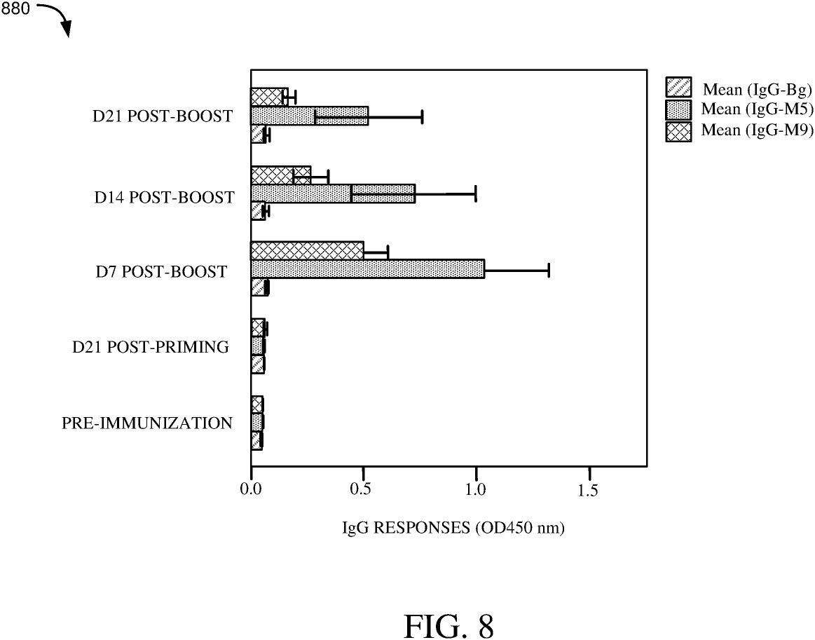

In some embodiments, the Qβ-V1V2-M5 conjugate was used as the primary immunogen and a Qβ-M9/M5 conjugate was used for the boost immunization in a single IV injection of 5.0 µg per mouse. To capture anti-M5 and anti-M9 antibodies, BSA-AsnGlcNAc2Man5 (BSA-M5) and BSA-AsnGlcNAc2Man9 (BSA-M9) conjugates (FIG. 7A) were coated using enzyme-linked immunosorbent assay (ELISA) at 1.0 µg per well, respectively. Of note, the M9-moiety was included in Qβ-M9/M5 to monitor potential selectivity of the Man5-specific boost of the secondary antibody responses. The vaccine strategy selectively triggers an active antibody response to the critical GNA-positive Man5-glyco-epotope, and results in detectable augmented secondary anti-Man5-IgG responses in the immunized mice. As shown by the graph of FIG. 8, the mice mounted significantly elevated anti-Man5 IgG responses on day 7 post-boosting immunization and, to the lesser extents, anti-Man9 IgG antibody responses.

FIG. 8 illustrates an example graph 880 showing induction of active IgG responses to oligomannose-based cryptic glyco-epitopes by an immunization strategy, in accordance with the present disclosure. Mouse sera were applied at 1:250 dilutions on ELISA plates coated with either BSA-Man5 (M5) or BSA-Man9 (M9), and the IgG antibodies were detected by an anti-mouse IgG-Fc-specific secondary antibody. Results were presented as mean IgG-signal of five B10 mice (OD450nm) (X-axis). Each error bar is constructed using 1 standard error from the mean.

Given that the ELISA capturing antigens, BSA-Man5 and BSA-Man9, differ only by their terminal non-reducing end sugar moieties, detection of a predominant IgG antibody responses by BSA-Man5 strongly suggests this two-step glycoconjugate immunization strategy selectively induced IgG responses to the GNA+-epitopes that are displayed by BSA-Man5, i.e., the Manα1,3Man and Manα1,6Man tri-saccharide moieties at the terminal non-reducing end of the M5-cluser (circled in various figures). These epitopes were, however, masked by the α1.2Man extension in the Man9-cluster. Somewhat surprisingly, detection of the weaker but significant levels of anti-Man9 IgG antibodies indicates that this vaccination also elicits responses to the shared epitopes of Man5 and Man9, such as the internal chain epitopes of high-mannoses. Given the fact that a broad search for induction of HIV-1 bnAbs targeting the 2G12-oligomannose-epitopes by active immunization has been unsuccessful for more than a decade, this result is surprising and highly encouraging.

Since GNA binds to and effectively neutralizes a broad-range of viruses, the GNA-epitope-based vaccine strategy can be generally effective in eliciting the GNA-like anti-oligomannose antibodies, and these antibodies offer the GNA model of bi-specific cross-linking heteroligation of virions (FIGS. 7C-7D) and hence function as broadly neutralizing antibodies against emerging viral pathogens.

Various embodiments were directed to GNA-epitope-based vaccine strategy applied to elicit the GNA-like antibodies in multiple stains of mice and identify if the vaccine-elicited anti-sera cross-react with a broad-range of viruses. The following example experimental embodiments were directed to identifying whether the glycoconjugates were effective in eliciting the GNA-like antibody responses in mouse models by testing the glycoconjugate vaccines using mice of different genetic backgrounds and determining the glycan-binding profiles and spectrum of virus-targeting of vaccine-induced antibody responses. Because anti-oligomannose antibody responses are often strongly influenced by a strain’s genetic background and IgH allotypes, vaccine responses in four strains of mice (2-4 months in age) were monitored, including BALB/c, C57BL/6, and their IgH-allotypic congenic strains, such as shown in Table 1 below.

TABLE 1

| | | |
|---|---|---|
| Strain | Background | IgH Allotype |
| BALB/c | BALB/c | IgH^{a} |
| C57BL/6 | C57BL/6 | IgH^{b} |
| B6.C20 | C57BL/6 | IgH^{a} |
| C.B-17 | BALB/c | IgH^{b} |

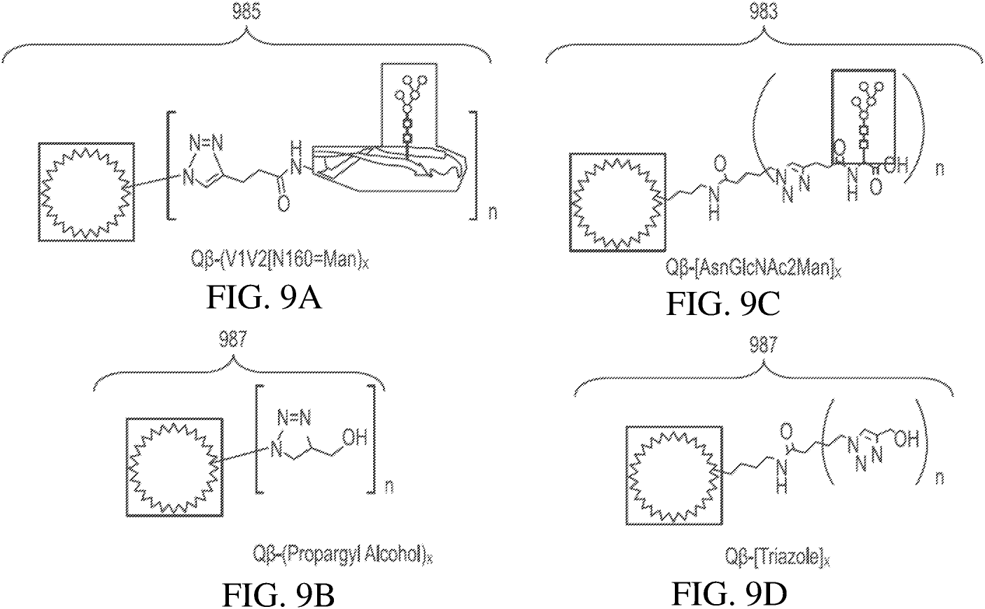

FIGS. 9A-9D illustrate example schematics of oligomannose-series of vaccine conjugates and a Qβ-control vector, in accordance with the present disclosure. M5-series of glycoconjugates, Qβ-(V1V2-M5)ₙ 985 and Qβ 1-(AsnGlcNAc₂Man5)ₙ 993 as shown by FIGS. 9A and 9C were applied to selectively induce anti-M5 antibody responses following the immunization strategy illustrated in FIG. 8. For each strain, 12 mice were immunized in the experimental group and 6 mice in the vector control group. The latter were immunized with corresponding M5-free vectors, as shown by Qβ-(Propargyl Alcohol)ₙ 989 and Qβ-(Triazole)ₙ 997 of FIGS. 9B and 9D.

In various embodiments, antigen microarrays were developed to determine whether the vaccine strategy induced GNA-like antibodies, characterize the mouse sera pre- and post-immunization can be performed.

For this purpose, glyco-antigen microarrays were constructed using a full panel of mannose cluster conjugates produced (e.g., FIG. 7A). Moreover, a collection of virus-derived glycoprotein preparations were spotted, including purified glycoproteins and viral lysates that are available from varies of sources (e.g., NIH AIDS Reagent Program, Microbix Biosystems Inc., Ontario, Canada, and Creative Diagnostics, NY, USA), to examine whether the vaccine induced antibodies to cross-react with a broad spectrum of viral antigens.

A versatile protein array substrate was used for microarray construction SuperEpoxy 2 Protein slides (ArrayIt Corporation, Sunnyvale, CA, USA). This substrate allows covalent coupling of protein carriers, leaving sugar moieties solvent-accessible for antibody recognition. Given the need to screen a large collection of anti-sera, approximately 1000 antigen microarrays were produced for the experimental embodiments.

As the above-described embodiments focused on N-glycan cryptic glyco-antigens, a bioarray of 388 features (four 96-well plates of antigen preparations) was sufficient to cover around 30 N-glycan autoantigens plus a panel of control antigens and to have at least two spotting concentrations and triplicate printing per dilution for each antigen. A practical 12-chamber-subarray design was used to construct such customized antigen microarrays for antibody screening. In this design, printing 100 SuperEpoxy slides generates 1200 microarrays.

The printed arrays were characterized using a panel of anti-oligomannose antibodies and lectins of defined glycan-binding specificities and/or virus-neutralizing activities, such as GNA, 2G12, and other mAbs with oligomannose binding specificities. Testing the printed antigen arrays by reagents of defined epitope specificities is a practical way to ensure the quality of microarrays. Antibody binding specificity can be correlated to viral neutralizing activity.

Some embodiments were directed to serological study. Standard microarray staining procedure was used to capture the antibody profiles from the serum of each mouse pre- and post-immunization and post-boosting at various time points and analyze the datasets using SAS Institute’s JMP Genomics software package (Cary, NC, USA). The microarray-identified positive antigens, e.g., those captured with significantly elevated antibody signal post-immunization or post-boosting, were further characterized by other immunoassays, such as mannose-cluster-specific ELISA, to quantitatively measure the anti-oligomannose antibodies, as shown by FIG. 8.

Some experimental embodiments were directed to virus-binding assays. The virus-binding assays were used to examine whether the vaccine-elicited anti-sera cross-react with a panel of emerging virus-pathogens. These include, but are not limited to, the MERS-CoV, SARS-CoV, SARS-CoV-2, ZIKV, DENV, WNV, HCMV, and HIV-1. For this purpose, the assays built on virus-infected cells or virus-particles for antibody detection (e.g., MERS-CoV EI 2604-1005 G, etc. from Euroimmun US, Inc. and other manufacturers) are used. Such assay platforms were designed to preserve the native viral glycoproteins, and the detected antibody titers often have good correlation with the virus-neutralizing activities of corresponding antibodies.

The experimental design allowed for determining whether genetic background and/or IgH allotype have any major impact on the oligomannose-specific anti-virus-antibody responses and if these anti-sera cross-react with a spectrum of emerging viral pathogens. Results further show B-cell responses to N-glycan cryptic autoantigens.

Various embodiments were directed to identifying mAbs and/or determining whether the mAbs elicited by the glycoconjugate vaccine exhibit GNA-like glycan-binding profile and epitope-recognition specificity and if the mAbs effectively neutralize a range of viruses expressing GNA-epitopes. For example, mAbs from vaccinated mice were captured to further characterize the GNA-like antibodies, including their glycan-binding specificities, model of virus-binding, efficacy of virus-neutralization, and IgV-molecular characteristics of these bnAbs. The mAbs can be captured by applying hybridoma technology and/or single B-cell antibody sequencing.

Some embodiments were directed to establishing hybridoma mAbs. Hybridomas can be generated using the vaccinated mice that mount GNA-like IgG responses. Selection of mice for cell-cell fusion to produce hybridomas was performed based on antibody profiles captured by antigen microarrays. Priority can be given to those producing anti-M5 IgG antibodies and antisera that cross-react with a broad spectrum of viruses. The established mAbs can be further characterized by antigen microarrays and other immunoassays.

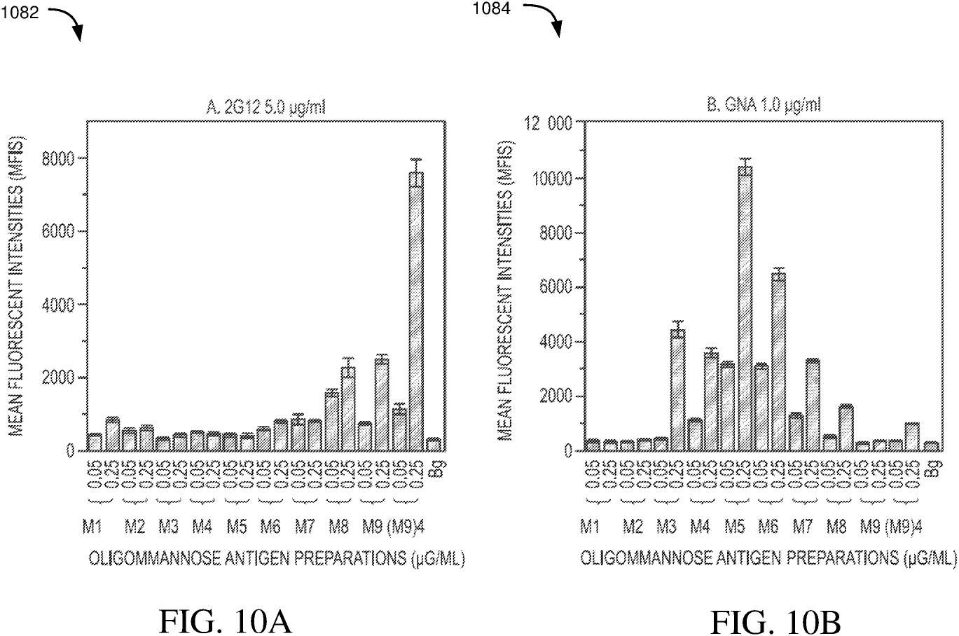

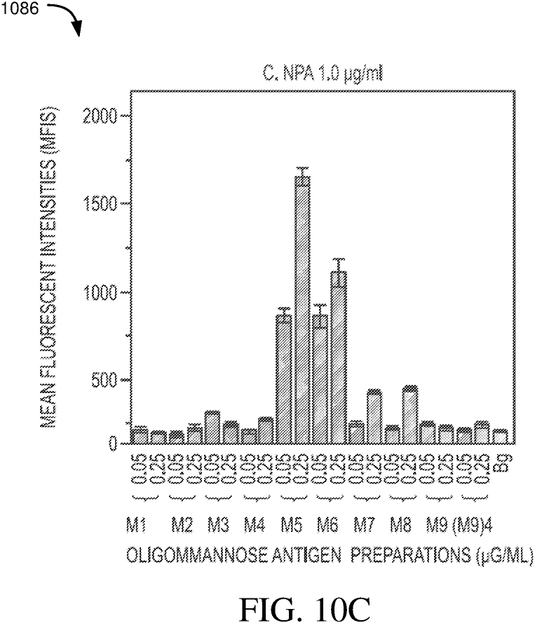

As previously described, some embodiments were directed to glycoconjugate production and characterization. A large-panel of oligomannose-protein conjugates of defined cluster configurations have been produced and applied to construct carbohydrate microarrays for probing virus-neutralizing epitopes. As previously described in associated with FIG. 7A, a panel of BSA-oligomannose conjugates (M1-9) and an HIV-gp120-mimiking conjugate, [(Man9GlcNAc2Asn)₄]-T-helper peptide conjugate ([(M9)₄]-TH) was used. The symbols M1 to M9 represent the neoglycoproteins Man1GlcNAc2Asn-BSA to Man9GlcNAc2-BSA. [(M9)₄]-TH mimics the native HIV spike epitopes recognized by 2G12 and GNA. A dominant tri-mannose GNA epitope is marked by the dashed circles. To determine whether these glycoconjugates preserve virus-neutralizing epitopes, the glycoconjugates were tested in ELISA (data not shown) and carbohydrate microarrays (e.g., FIGS. 10A-10C) using virus-neutralizing agents. Key probes include antibodies 2G12, GNA, and NPA. The two lectins, NPA and GNA, are specific for terminal Manα1,6Man moieties and Manα1,3Man/Manα1,6Man linkages respectively, while 2G12 targets the high-mannose patch on the envelop glycoprotein gp120 of HIV by recognizing the terminal Manα1,2Man linkages in the oligomannose cluster.

FIGS. 10A-10C illustrate example binding profiles for mannose-reactive proteins 2G12, GNA, and NPA to BSA-oligomannose conjugates, in accordance with the present disclosure. More specifically, FIGS. 10A-10C are graphs 1082, 1084, 1086 illustrating the binding profiles of mannose-reactive proteins 2G12, GNA, and NPA to BSA-oligomannose conjugates, which are sometimes herein interchangeably referred to as the “glycoconjugates” or the “oligomannose-BSA conjugates”. The glycoconjugates were spotted at 0.05 µg/µL and 0.25 µg/µL. Glycan-binding activities of antibody/lectin are shown as means of fluorescent intensities (MFIs) of triplicate micro-spots. Each error bar is constructed using one standard deviation from the mean of triplicate detections. The background (Bg) signal served as the negative control.

Using the above-described key probes to scan carbohydrate microarrays, it was identified that the intensity of the 2G12 recognition of the oligomannoses-BSA conjugates roughly corresponded to the number of Manα1,2 linkages present. The affinity of 2G12 for the oligomannose-BSA conjugates decreased for conjugates bearing fewer Manα1, 2 linkages. The gp120-mimiking [(M9)4]-TH exhibited very strong binding, while among the BSA conjugates, M9- and M8-BSA showed the best recognition. The far lower binding of the BSA conjugates as compared to [(M9)4]-TH can be attributed to the mode of oligomannose presentation. The high-mannose patch of gp120 and the tetravalent [(M9)4]-TH conjugate present the oligomannoses in a dense, clustered arrangement optimal for 2G12 binding. In the case of lectin binding, the oligomannose-BSA conjugates with higher access to the α1, 6-linked mannose generally bound more strongly to NPA, with compounds Man5-BSA and Man6-BSA displaying the highest affinity. Recognition of the BSA conjugates by GNA appears to be dependent upon the accessibility of the target Manα1, 3-Man linkages. GNA had little affinity for compound M9-BSA, however, recognition improved as access to the core Manα1,3Man linkages became more available. It follows that the two smallest paucimannose constituents (M1 and M2) had no affinity for GNA as they both lack Manα1,3Man linkages.

As described above, successful construction of carbohydrate microarrays was performed to display a large-panel of viral protein-free glycoconjugates and reconstructed the virus-neutralizing epitopes that are specifically recognized by 2G12, GNA, or NPA. Although these virus-neutralizing epitopes are composed of oligomannoses, they differ in mannosyl cluster configurations. Unlike 2G12-epitopes, which is formed by the compacted high-density Man9-clusters, GNA- and NPA-epitopes are overlapping and share the tri-mannose core as highlighted in FIG. 7A.

Some experimental embodiments were directed to construction of immunogenic vaccines using GNA-positive oligomannoses. Vaccine conjugates were produced by coupling oligomannoses to the immunologically potent carrier proteins to enhance immune responses to glyco-epitopes (e.g., sugar moieties). Specifically, different carrier proteins, such as the icosahedral capsids of the bacteriophage Qβ or KLH for glycoconjugation was applied to produce a potential vaccine.

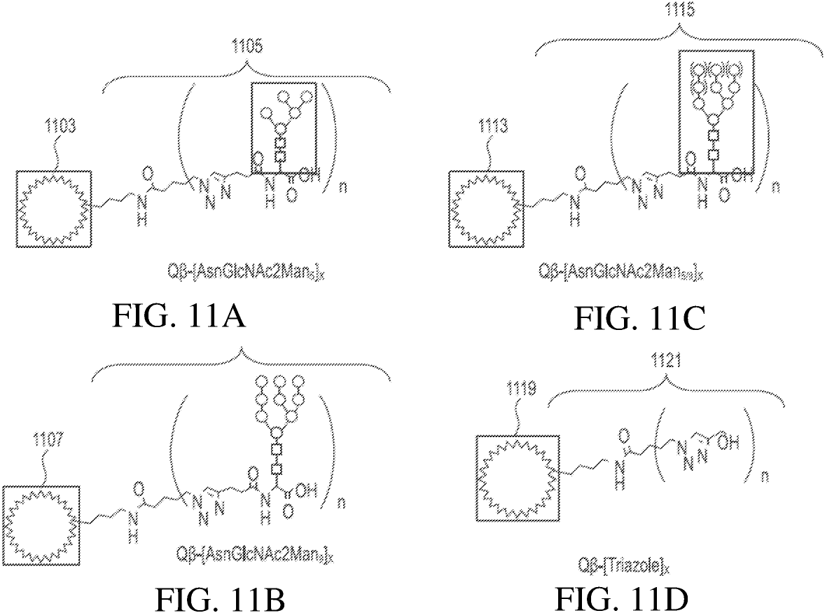

FIGS. 11A-11D illustrate example schematics of oligomannose-series of vaccine conjugates and a Qβ-control vector, in accordance with the present disclosure. More particularly, FIGS. 11A-11D show schematic of Qβ-series of vaccine conjugates, Qβ 1103 -(AsnGlcNAc₂Man5)ₙ 1105 (herein generally referred to as “Qβ-M5”), Qβ 1107 -(AsnGlcNAc₂Man9)ₙ 1109 (herein generally referred to as “Qβ-M9”), and Qβ-1113 (AsnGlcNAc₂Man5)/(AsnGlcNAc₂-Man9) 1115 (herein generally referred to as “QP-MS-9”). In the latter, a GNA+ carbohydrate, Man5, and an intercellular adhesion molecule-3-grabbing non-integrin (DC-SIGN) ligand, Man9 were co-conjugated to surface of Qβ. The control conjugate included Qβ 1119 -(Triazole)ₙ 1121. As virus-like particles (VLPs), the Qβ displays clusters of these epitopes on the surface to offer cross-linking stimulation of DCs and B-cells. Moreover, QP-VLPs are packaged with E. coli RNA, which serves as a built-in adjuvant through the activation of toll-like receptor (TLR7/8). Thus, the resulting neoglycoconjugate, QP-MS-9, offers a multivalent-display of Man5 and Man9 epitopes and has the unique capacity to target DCs and co-stimulate TLRs to activate DCs.

Some experiments were directed to vaccine strategies to enhance anti-oligomannose antibody responses. Vaccine strategies were studied using a panel of synthetic glycoconjugates and multiple ways to enhance oligomannose-specific antibody responses in mouse models were explored.

Table 2 illustrates example of some experimental designs. In this set of experiments, three vaccine combinations were tested. In group 1, Balb/c mice (IgHa) were immunized with two structurally irrelevant antigens D33-O-core and Qβ-M5-9. In group 2, the two glycoconjugates, KLH-M9 and Qβ-M5-9, share M9-epitopes but differ in the protein carrier molecules. By contrast, the two antigens in group III, Qβ-M5 and Qβ-M5-9, have an identical carrier and commonly express M5-epitopes. In various embodiments, it was then examined whether these conjugates elicit memory B-cell responses to oligomannose epitopes and which vaccine strategy facilitates induction of oligomannose-specific IgG responses. In some experiments, a dosage range of between 0.2 mg/kg to 0.3 mg/kg of the glycoconjugates identified in Table 2 may be provided to mice. In some experiments, a dose of 0.25 mg/kg of the glycoconjugates identified in Table 2 was provided to mice and/or for each dosage. However, examples are not so limited.

Splenic B-cell responses in mouse models were examined to offer a rapid test of potential immunogenicity of these glycoconjugates (Table 2). Specifically, mice were triggered with a single dose IV injection of a soluble glycoconjugate. Of note, no adjuvant was applied in both primary immunization and the secondary boost immunization. Then immune responses were monitored using blood samples.

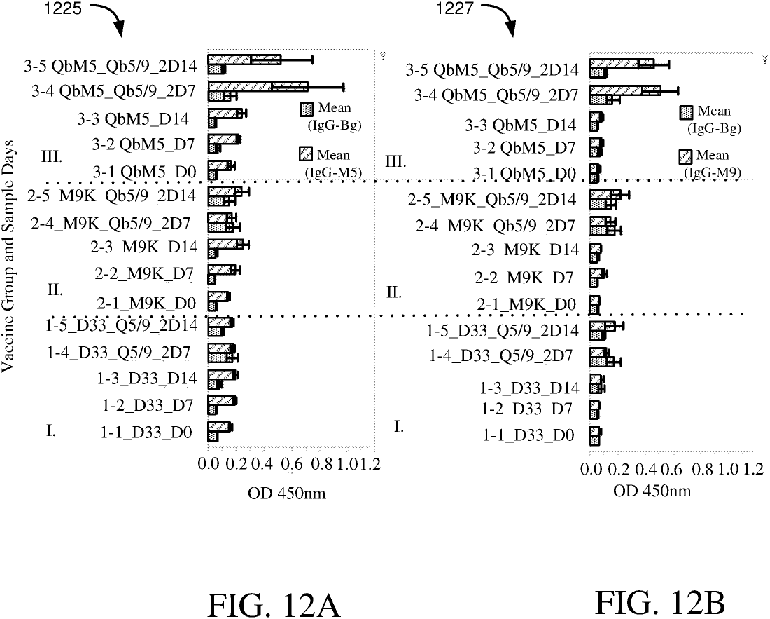

FIGS. 12A-12B illustrate examples induction of active IgG responses to oligomannose-based virus-neutralizing epitopes by an immunization strategy, in accordance with the present disclosure. More particularly, the graphs 1225, 1227 in FIGS. 12A-12B summarize representative results of serum IgG responses to M5 or M9 in the three vaccine groups listed in Table 2. Here, mouse serum was applied at 1:250 dilutions on ELISA plates coated with either BSA-Man5 (M5) or BSA-Man9 (M9).

TABLE 2

| | | | | |
|---|---|---|---|---|
| Group | Mice | Sex | Day 0, Priming | Day 30, Boosting |
| I | Balb/C-1 | Female | D33-O-core, 10ug, IV | QP-M5-9, 5ug, IV |
| I | Balb/C-2 | Female | D33-O-core, 10ug, IV | QP-M5-9, 5ug, IV |
| I | Balb/C-3 | Female | D33-O-core, 10ug, IV | QP-M5-9, 5ug, IV |
| I | Balb/C-5 | Female | D33-O-core, 10ug, IV | QP-M5-9, 5ug, IV |
| I | Balb/C-5 | Female | D33-O-core, 10ug, IV | QP-M5-9, 5ug, IV |
| II | Balb/C-6 | Female | KLG-M9, 5ug, IV | QP-M5-9, 5ug, IV |
| II | Balb/C-7 | Female | KLG-M9, 5ug, IV | QP-M5-9, 5ug, IV |
| II | Balb/C-8 | Female | KLG-M9, 5ug, IV | QP-M5-9, 5ug, IV |
| II | Balb/C-9 | Female | KLG-M9, 5ug, IV | QP-M5-9, 5ug, IV |
| II | Balb/C-10 | Female | KLG-M9, 5ug, IV | QP-M5-9, 5ug, IV |
| III | Balb/C-11 | Female | Qβ-Gn-M5, 5ug, IV | QP-M5-9, 5ug, IV |
| III | Balb/C-12 | Female | Qβ-Gn-M5, 5ug, IV | QP-M5-9, 5ug, IV |
| III | Balb/C-13 | Female | Qβ-Gn-M5, 5ug, IV | QP-M5-9, 5ug, IV |
| III | Balb/C-14 | Female | Qβ-Gn-M5, 5ug, IV | QP-M5-9, 5ug, IV |
| III | Balb/C-15 | Female | Qβ-Gn-M5, 5ug, IV | QP-M5-9, 5ug, IV |

In the assays, a BSA-Man5 or a BSA-Man9 conjugate was coated in ELISA. Because these conjugates differ from the immunogens in the protein carriers, this assay detects oligomannose-specific antibodies. The detected IgG antibodies were presented as mean of five Balb/c mice in each given time point. Each error bar is constructed using 1 standard error from the mean. The days post-primary immunization is labeled as D7 and D14, respectively. For the secondary immune responses, days post-boosting is marked as 2D7 and 2D14, accordingly.

Group I represents a “hetero-antigen” immunization design with D33-O-cores for priming and a Qβ-M5/M9 conjugate for the boost immunization. Both antigens have DC-SIGN-targeting activity, which may modulate immune responses. However, this vaccine combination did not trigger significant increase in anti-Man5 IgG activity; only marginal levels of anti-Man9 IgG antibodies were detected day 14 post-boosting with Qβ-M5/M9.

Group II is a “hetero-carrier” immunogen pair, composed of KLH-M9 and Qβ-M5-9. Although both KLH and Qβ are highly immunogenic and are potent T-cell activators, this combination appears not very effective in triggering anti-Man5 or anti-Man9 IgG responses.

Group III is a “homo-carrier” immunogen pair composed of Qβ-M9 and Qβ-M5-9. Interestingly, this vaccine strategy elicited highly significant IgG responses to both Man5 and Man9. The peak of serum IgG responses appeared at day 7 post-boosting with QP-M5-9.

Qβ-M5-9 was further examined to determine if it is effective in enhancing anti-oligomannose responses in the group I and II mice. These mice had already received one dose of Qβ-M5-9 at day 30. Mice were boosted again with Qβ-M5-9 and found that virtually all mice produced significant levels of anti-Man5 and anti-Man9 IgG after the second dose of “homo-carrier” glycoconjugates.

The Qβ-V1V2-M5 conjugate was also administered as the primary immunogen and a Qβ-M9-M5 conjugate for the boost immunization in group of B10.Ai mice (IgH^{b}) and elicited a dominant anti-M5 IgG response as observed in Balb/c (IgH^{a}). Thus, the Qβ-based “homo-carrier” pair design illustrates potential as an effective strategy for eliciting oligomannose-specific IgG responses in mouse models, including IgH^{a}- and IgH^{b}-allotypes of mice.

Various experimental embodiments were directed to establishment of a large hybridoma library for screening anti-oligomannose mAbs. With the significant progress in mouse immunization, vaccinated mice were used to produce hybridomas and screened for oligomannose-specific clones (FIGS. 13A-13B).

FIGS. 13A-13B illustrate example mAbs that may recognize distinct epitopes of high-mannose antigens, in accordance with the present disclosure. FIG. 13A provides example schematics of potential epitopes formed by terminal sugar moieties of am oligomannose chain of Man5, as shown by 1331, and an oligomannose chain of Man9, as shown by 1333, with potential epitopes formed by the internal sugar moieties highlighted. FIG. 13B is a graph 1335 illustrating example anti-oligomannose mAbs.

To maximize the success rates in obtaining a diverse panel of anti-oligomannose mAbs, a collection of hybridomas was established with fusion cells and corresponding culture supernatants frozen down. For each hybridoma fusion experiment, the splenic B-cells from one vaccinated mouse was used. By processing around 30 fusion experiments, a large collection of hybridomas were accumulated and screened by glycan-specific ELISA and/or carbohydrate microarrays to identify hybridomas producing anti-oligomannose antibodies.

More particularly, FIGS. 13A-13B show epitope-mapping of a few clones. In this assay, hybridoma culture supernatants were applied on ELISA plate coated with BSA-MS or BSA-M9. The captured oligomannose-specific antibodies were revealed with alkaline phosphatase-conjugated secondary anti-mouse antibodies. As illustrated, initial screening has identified three types of anti-oligomannose hybridoma mAbs, i.e., M5-specific, M9-specific, and M5/9-specific types. Thus, hybridomas can be used for screening the GNA-like mAbs.

Some embodiments were directed to a use case specific to coronaviruses’ sugar shield as vaccine candidate and therapeutic targets. As previously mentioned, due to the genetic and proteomic diversities of viral pathogens, establishing versatile anti-viral vaccines or therapeutic agents is highly challenging. Carbohydrate antigens represent an important class of immunological targets for vaccine development and immunotherapy against microbial infections. Also disclosed herein are oligomannoses as immunogenic carbohydrate moieties of CoVs (including SARS-CoV, SARS-CoV-2, and other CoVs) and establishment of oligomannose-specific mAbs as candidate therapeutic agents for broadly CoV-neutralization.

Using the novel glycoconjugates, high-throughput carbohydrate microarrays, and other oligomannose-specific immunoassays described above, CoV vaccine responses can be characterized. Novel glycoconjugates can be constructed and applied that are free of any CoV-protein element as vaccines and successfully establish a large library of hybridomas. Initial screening of this library has identified a panel of hybridoma mAbs with oligomannose-binding specificities as key candidates for use as therapeutic antibodies.

A common feature of different CoVs is that their S glycoproteins are densely decorated by N-linked glycans protruding from the surfaces of the virions. The SARS-CoV-2-S comprises 22 N-linked glycosylation sites, and 16 of them were resolved in the cryo-electron microscopy (cryoEM) map as glycosylated. By comparison, SARS-CoV-S possesses 23 N-linked glycosylation sites with at least 19 of them confirmed to be glycosylated. Twenty out of 22 SARS-CoV-2-S N-linked glycosylation sites are conserved in SARS-CoV-S. Specifically, 9 out of 13 sites in the S1 subunit and all 9 sites in the S2 subunit are conserved among SARS-CoV-2-S and SARS-CoV-S. CoVs may overexpress the high-mannose type since CoV virions are likely matured in and directly bud from the endoplasmic reticulum-Golgi intermediate compartment without further editing by the Golgi-residential glyco-enzymes.

As previously described, carbohydrate panels can be used for exploring the immunogenic sugar moieties recognized by host immune systems to mount antibody responses. Unlike a conventional S glycoprotein immunoassay that detects the sum of anti-protein and anti-oligomannose antibodies, carbohydrate panels can be designed to present either pure carbohydrate moieties or glycoconjugates lacking S protein components and, thereby, can be used to decipher anti-oligomannose and anti-protein antibodies for a given immunogen or pathogen. Characterizing a SARS-CoV-2 vaccine response or COVID-19 patients’ serological response using carbohydrate panel can be used to verify whether SARS-CoV-2 is also decorated with glyco-determinants as immunological targets.

Additionally, such experimental embodiments can be used to identify glyco-immunological information to guide development of glycoconjugate vaccines and therapeutic antibodies to target the sugar shield of SARS-CoV-2 and other unexpected CoVs with human outbreak potential. The glycoconjugate vaccines without any CoV protein component may have the unique advantage of avoiding undesired vaccine responses to the S-protein epitopes that were non-neutralizing but elicited the antibody-dependent enhancement of infectivity and severe Th2-type lung immunopathy observed during SARS-CoV vaccine development.

As used herein, an immunogenic vaccine composition includes and/or refers to a composition that is administered to stimulate an immune system of a subject and/or to produce an immune response to a viral pathogen. Example immunogenic vaccine compositions include high mannose compositions, such as a glycoconjugate. A glycoconjugate includes and/or refers to one or more glycans (e.g., oligomannoses) linked to another compound, such as carrier protein. For example, the glycoconjugate can include clusters (e.g., linear or branches) of oligosaccharides linked to the carrier protein. Oligosaccharides may include monosaccharides, disaccharide, trisaccharide, and polymer of mannose units, e.g., mannosyl or sugar moieties such as Man1-Man9 and combinations thereof. ManX (e.g., Man1, Man2, etc.) refers to a glycan that includes mannose units, where the number refers to the number of mannose units in the glycan. A viral pathogen includes and/or refers to an organism that can produce a viral infection in a subject. A subject, as used herein, includes and/or refers to a host organism that can be infected by a viral pathogen. Example subjects include mammals, such as humans. B-cells, which are sometimes referred to as B lymphocytes, include and/or refer to a type of white blood cell of the lymphocyte subtype. B-cells are a function of the immune system and can secrete antibodies. Hybridomas include and/or refer to a hybrid cell used as the basis for the production of antibodies, such as for diagnostic or therapeutic use. An antibody includes and/or refers to a protein used by the immune system to detect, neutralize, and/or kill various target cells, such as viral pathogens, which may be harmful to the host organism. As used herein, mAbs include and/or refer to antibodies produced by a single clone of a cell or cell line and can include identical molecules. Oligomannose-specific mAbs include and/or refer to mAbs that are specific to an oligomannose (e.g., recognize an epitope of oligomannose). An anti-oligomannose antibody includes and/or refers to an antibody that is specific to an oligomannose (e.g., recognizes an epitope of an oligomannose). An anti-oligomannose bnAb is a bnAb that is specific to an oligomannose (e.g., recognizes an epitope of an oligomannose) and which may neutralize a target protein. As used herein, an antibody specific to an oligomannose or other epitope includes and/or refers to an antibody that recognizes (e.g., binds) to the epitope. An epitope, as used herein, includes and/or refers to an antigenic determinant. An epitope can be part of the antigen that is recognized by the immune system and specially binds to antibodies, B-cells and/or other immune cells. A glyco-epitope includes and/or refers to an epitope of a glycan, such as an epitope of an oligomannose, sometimes referred to as a sugar moiety. In some embodiments, the glyco-epitope is a terminal part of the oligomannose chain (e.g., a terminal sugar moiety) and/or is a side chain or internal part of the oligomannose chain (e.g., a side chain or internal sugar moiety). An oligomannose, as used herein, includes and/or refers to a glycan (e.g., a polysaccharide or oligosaccharide) that is an oligomer composed of mannose units. Example oligomannoses include pure oligomannoses, as well as hybrid and/or complex forms of glycans containing oligomannoses, which are sometimes herein referred to as oligomannosyls. As such, oligomannose, as used herein, is not limited to pure forms of oligomannose. Mannose includes and/or refers to a sugar monomer of the aldohexose series of carbohydrates. A carrier protein includes and/or refers to a protein that is linked to other molecules, such as oligomannose chains exhibiting the glyco-epitopes.

Various embodiments are implemented in accordance with the underlying Provisional Application Ser. No. 63/009,045, entitled “Immunogenic Sugar Moieties as Versatile Vaccine Candidates,” filed Apr. 13, 2020, and including the Appendix to the Specification, to which benefit is claimed and which are fully incorporated herein by reference for their general and specific teachings. For instance, embodiments herein and/or in the Provisional Application can be combined in varying degrees (including wholly). Reference can also be made to the experimental teachings and underlying references provided in the underlying Provisional Application. Embodiments discussed in the Provisional Application are not intended, in any way, to be limiting to the overall technical disclosure, or to any part of the claimed disclosure unless specifically noted.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method, comprising:
administering an immunogenic vaccine composition to a subject, the immunogenic vaccine composition comprising a glycoconjugate; and
in response to the immunogenic vaccine composition, inducing production of anti-oligomannose antibodies in the subject and thereby eliciting an immune response to a viral pathogen in the subject.

2. The method of claim 1, further comprising triggering splenic B-cell responses and co-activating C-type lectin dendritic cell-specific intercellular adhesion molecule-3-grabbing non-integrin (DC-SIGN)-mediated dendritic cell responses in response to the immunogenic vaccine composition, thereby inducing the production of the anti-oligomannose antibodies and eliciting the immune response.

3. The method of claim 1, wherein the anti-oligomannose antibodies include broadly neutralizing antibodies (bnAbs) that recognize surface-exposed oligomannoses expressed by the viral pathogen.

4. The method of claim 1, wherein administering the immunogenic vaccine composition includes injecting a soluble form of the immunogenic vaccine composition to the subject.

5. The method of claim 1, further comprising administering an additional immunogenic vaccine composition to the subject, wherein the additional immunogenic vaccine composition comprises one of:
the glycoconjugate of the immunogenic vaccine composition; and
a different glycoconjugate from the glycoconjugate of the immunogenic vaccine composition.

6. The method of claim 1, wherein the glycoconjugate includes terminal glyco-epitopes recognized by the anti-oligomannose antibodies.

7. The method of claim 1, wherein the glycoconjugate includes internal chain or side-face glyco-epitopes recognized by the anti-oligomannose antibodies.

8. The method of claim 1, further comprising eliciting the immune response in the subject against the viral pathogen, the viral pathogen including at least one of Middle East respiratory syndrome coronavirus (MERS-CoV), severe acute respiratory syndrome (SARS)-CoV, SARS-CoV-2, Zika virus (ZIKV), Dengue virus (DENV), West Nile virus (WNV), human cytomegalovirus (HCMV), and Human immunodeficiency virus (HIV-1).

9. The method of claim 1, wherein the glycoconjugate includes a carrier protein linked to oligomannose chains having a plurality of glyco-epitopes recognized by the anti-oligomannose antibodies.

10. The method of claim 1, further comprising identifying oligomannoses for the glycoconjugate by screening the viral pathogen or a neutralizing agent that reacts with the viral pathogen against an array of a plurality of different oligomannoses.

11. The method of claim 1, further comprising:
after eliciting the immune response, producing hybridomas using antibody producing cells obtained from the subject;
screening the hybridomas for the anti-oligomannose antibodies using an array of a plurality of different oligomannoses; and
generating oligomannose-specific monoclonal antibodies (mAbs) from at least one of the anti-oligomannose antibodies.

12. The method of claim 1, wherein eliciting the immune response includes broadly providing prevention from infection or an immune response to different viral pathogens by the production of the anti-oligomannose antibodies in the subject, wherein each of the different viral pathogens express and surface-expose oligomannoses.

13. A method, comprising:
administering a first immunogenic vaccine composition to a subject;
administering a second immunogenic vaccine composition to the subject, the first and second immunogenic vaccine compositions each comprising a glycoconjugate having glyco-epitopes; and
in response, inducing production of anti-oligomannose broadly neutralizing antibodies (bnAbs) that recognize the glyco-epitopes and thereby eliciting an immune response to a viral pathogen that expresses surface-exposed oligomannoses associated with the glyco-epitopes in the subject.

14. The method of claim 13, wherein administering the first and second immunogenic vaccine compositions includes:
providing a first intravenous injection to the subject that includes a soluble form of the first immunogenic vaccine composition, and in response, resulting in at least one of triggering splenic B-cell responses and co-activating the C-type lectin dendritic cell-specific intercellular adhesion molecule-3-grabbing non-integrin (DC-SIGN)-mediated dendritic cell responses; and
providing a second intravenous injection to the subject that includes a soluble form of the second immunogenic vaccine composition, and in response, boosting splenic B-cell responses and C-type DC-SIGN-mediated dendritic cell responses, thereby inducing the production of the anti-oligomannose bnAbs and eliciting the immune response.

15. The method of claim 14, wherein the first immunogenic vaccine composition and the second immunogenic vaccine composition include the same glycoconjugate having the same terminal glyco-epitopes.

16. The method of claim 14, wherein the first immunogenic vaccine composition and the second immunogenic vaccine composition include different glycoconjugates having different terminal glyco-epitopes from another.

17. The method of claim 13, wherein eliciting the immune response includes broadly providing preventative infection from or immune response to different viral pathogens by the production of the anti-oligomannose bnAbs in the subject, wherein each of the different viral pathogens express and surface-expose the oligomannoses.

18. The method of claim 13, wherein eliciting the immune response includes providing preventative immune response to at least one of Middle East respiratory syndrome coronavirus (MERS-CoV), severe acute respiratory syndrome (SARS)-CoV, SARS-CoV-2, Zika virus (ZIKV), Dengue virus (DENV), West Nile virus (WNV), human cytomegalovirus (HCMV), and Human immunodeficiency virus (HIV-1).

19. A method comprising:
producing hybridomas using antibody producing cells obtained from a subject treated with an immunogenic vaccine composition, the immunogenic vaccine composition comprising a glycoconjugate designed to induce production of anti-oligomannose antibodies that recognize surface-exposed oligomannoses;
screening the hybridomas for the anti-oligomannose antibodies using an array of a plurality of different oligomannoses; and
generating oligomannose-specific monoclonal antibodies (mAbs) from at least one of the anti-oligomannose antibodies.

20. The method of claim 18, wherein generating the oligomannose-specific mAbs includes generating mAbs specific to one or more of Man1, Man2, Man3, Man4, Man5, Man6, Man7, Man8, and Man9.
